(19) United States
(12) Patent Application Publication
(10) Pub. No.: US 2014/0363405 A1
(43) Pub. Date: Dec. 11, 2014

(54) METHOD FOR OBTAINING MAB-LIKE CELLS AND USES THEREOF

(71) Applicant: UCL Business PLC, London (GB)

(72) Inventors: Giulio Cossu, London (GB); Francesco Saverio Tedesco, London (GB)

(73) Assignee: UCL Business PLC, London (GB)

(21) Appl. No.: 14/373,399

(22) PCT Filed: Jan. 18, 2013
(86) PCT No.: PCT/GB2013/050112

§ 371 Date: Jul. 21, 2014

Related U.S. Application Data
(60) Provisional application No. 61/588,269, filed on Jan. 19, 2012.

(51) Int. Cl. A61K 35/54

(52) U.S. Cl. CPC A61K 35/54
USPC 424/93.7; 435/377

(57) ABSTRACT

The invention relates to the derivation of mesoangioblast-like (MAB-like) cells from pluripotent cells such as induced pluripotent (IPS) and embryonic stem (ES) cells, to cells obtained thereby and to medical uses of such cells, in particular in the treatment of muscular dystrophies.

### FIELD OF THE INVENTION

Human induced pluripotent stem cells from limb-girdle muscular dystrophy 2D generate mesoderm stem/progenitor cells for ex vivo gene therapy.

### BACKGROUND OF THE INVENTION

Mesoangioblasts (MABs) are stem/progenitor cells derived from a subset of pericytes expressing alkaline phosphatase. They have been shown to ameliorate muscular dystrophies (currently incurable diseases) in different animal models and are now undergoing clinical experimentation for Duchenne muscular dystrophy. WO2007/093412 (40) provides details of this approach. Methods for isolating and expanding mesoangioblasts are also disclosed in WO2003/095631 (41).

### SUMMARY OF THE INVENTION

We show here that patients affected by limb-girdle muscular dystrophy 2D (LGMD2D, characterized by α-sarcoglycan deficit) have a reduction of this subset of pericytes and hence mesoangioblast could not be derived for cell therapy. Therefore, we reprogrammed LGMD2D fibroblasts and myoblasts to induced pluripotent stem cells (iPSCs) and developed a protocol for the derivation of mesoangioblast-like cells from them. These cells can be expanded and genetically corrected with a muscle-specific lentiviral vector expressing human α-sarcoglycan. Upon transplantation into ad hoc generated α-sarcoglycan-null immunodeficient mice, they generate myofibers expressing α-sarcoglycan. This approach may be useful for muscular dystrophies that show a reduction of resident progenitors and provides evidence of pre-clinical safety and efficacy of disease-specific iPSCs.

We have also extended our method of deriving MABs from human iPS cells to deriving MABs from human embryonic stem cells (ES cells).

Accordingly, the invention provides:
Method for obtaining mesoangioblast (MAB)-like mesodermal stem/progenitor cells from pluripotent stem cells comprising the following steps:
(a) dissociating colonies of said cells to a first single cell suspension;
(b) seeding the first single cell suspension on a solid support coated with a cell culture substrate with appropriate culture medium and temperature in a low O₂ atmosphere to get a first cell culture;
(c) dissociating the cell culture to get a second single cell suspension;
(d) seeding the second single cell suspension as in step b) to get a second cell culture;
   wherein steps (c) and (d) are optionally repeated.
   The invention also provides:
   Mesoangioblast (MAB)-like mesodermal stem/progenitor cells obtainable or obtained with the method of the invention.
   Mesoangioblast (MAB)-like mesodermal stem/progenitor cells according to the invention for use as a medicament.
   Method of treatment of a muscular dystrophy comprising the administration of a therapeutically effective amount of the cells according to the invention to a subject in need thereof.

### BRIEF DESCRIPTION OF THE DRAWINGS

FIG. 1. Schematic representation of the iPSC-based gene and cell therapy strategy.

FIG. 2. Reduction of AP+ pericytes in LGMD2D.

(A) The histograms show FACS analysis for AP and CD56 of six skeletal muscle cell preparations (one healthy donor and five LGMD2D patients; the first two histograms refer to cells obtained from biopsies). (B) The images depict in vitro skeletal muscle differentiation of the samples in (A) (MyHC: myosin heavy chain; scale bar: 80 μm). (C) Hematoxylin & eosin plus enzymatic AP staining (blue) of skeletal muscle sections from the patients shown in (A) and (B), indicating reduced number of AP+ cells (arrows indicate examples in one LGMD2D patient and its control; scale bar: 100 μm; picture in the lower row contain magnifications from the fields in the white rectangles). (D) Bar graph quantifying the reduction shown in (C) versus matched healthy controls (white bars (CT) placed on the right side of each patient (black bar); *** P<0.0005; unpaired t-test). (E) Histology and quantification of AP+ pericyte reduction in Sgca-null mice compared with matched WT controls at two different ages (right hand pictures are magnifications of the fields contained in the rectangles; scale bar: 100 μm; *** P<0.0005, unpaired t-test).

FIG. 3. Generation and characterization of HIDEMs derived from healthy donors.

(A) Scheme of the differentiation protocol from starting cells to HIDEMs. Details in Materials and Methods. (B) Phase contrast morphology and (C) AP staining of HIDEMs and human MABs at the same passage in culture showing comparable features (scale bar: 80 μm). (D) Growth curves of two HIDEM lines and control human MABs showing comparable proliferation rate. (E) Representative (n=3) gel containing a ladder of PCR products showing telomerase activity of pre-reprogramming fibroblasts (F), iPSCs (i) and relative HIDEMs (H) done by telomeric repeat amplification protocol (TRAP). Virus-free HIDEMs do not have a fibroblast lane because they were purchased as iPSCs. As a control, primary MEFs (M) and a negative control (CT-) are shown. (F) Karyotype analysis showing correct ploidy in two representative HIDEM populations (#1: 46,XX; #3: 46, XY) after >20 population doublings after derivation from iPSCs. (G) Immunofluorescence analysis for the reprogramming factors and for Nanog showing their absence in HIDEMs (scale bar: 50 μm). Insets show positive controls: iPSC colonies for SOX2, OCT4 and Nanog, and HeLa cells for cMYC. (H) Bar graph depicting a representative example of a quantitative real-time PCR analysis of total and exogenous SOX2, OCT4 and KLF4 transcripts from iPSCs (black bars) to HIDEMs (green bars), including an intermediate immature HIDEM population (red bars; that had a premature stop in the differentiation protocol).

FIG. 4. Molecular signature and skeletal muscle differentiation of HIDEMs.

(A) FACS analysis of undifferentiated iPSCs, partially differentiated (immature) HIDEMs, differentiated (mature) HIDEMs and control adult human MABs (hMABs) demonstrating down-regulation of pluripotency markers (SSEA4 and AP) and up-regulation of human MABs markers (in red). (B) Affymetrix GeneChip microarray analysis showing unsupervised hierarchical clustering of HIDEMs, MABs, ESCs, fibroblasts (FIB), endothelial cells (END), mesenchymal stem cells (MSC), smooth muscle (SM)-derived cells, neural progenitors (NPC) and iPSCs. Data were meta-analyzed as described in the Supplementary Material (See Example 2). (C) Co-culture assay of GFP positive HIDEMs and C2C12 myoblasts: fluorescent (green) myotubes are present in vitro after 3 days in differentiation medium (scale bar: 70 μm. (D) Immunofluorescence of the same co-culture assay shown in (C) depicting a GFP positive myotube containing three HIDEM nuclei (arrows; scale bar: 30 μm; see also FIG. 7D). The bar graph quantifies contribution of human nuclei to myotubes. (E) Immunofluorescence showing early in vitro myogenic differentiation of HIDEMs two days after tamoxifen-induced MyoD-ER over-expression (scale bar: 50 μm). (F) Myogenic conversion of two representative lines five days after tamoxifen administration (scale bar: 100 μm). (G) RT-PCR analysis of SGCA and myogenic regulatory factors (MYOD and MYOGENIN) in terminally differentiated MyoD-ER-transduced HIDEMs (H5V is an endothelial cell line shown as a negative control).

FIG. 5. Reprogramming of LGMD2D cells to iPSCs and derivation of HIDEMs.

(A) Representative morphology of a LGMD2D cellular population obtained after culture of a skeletal muscle biopsy (scale bar: 50 μm). (B) Reprogramming of LGMD2D cells to iPSCs (OKS±M: 2/4 lines were not transduced with cMYC). The upper pictures show morphology, AP staining and NANOG expression of LGMD2D iPSCs (white scale bar: 0.9 mm; black scale bar: 0.8 mm). The following panel shows a teratoma formation assay done with the upper colonies (see Supplementary Material (See Example 2) for details): the top two pictures show the mass before and after resection from a NOD/scid mouse; the image in the center is an hematoxylin and eosin staining of a section of the upper mass showing examples (inside boxes) of differentiation into tissues of the three germ layers (scale bar: 250 μm). (C) LGMD2D iPSC-derived HIDEMs. The top two pictures depict morphology and AP staining of the cells (scale bar: 50 μm), followed by three images showing correct karyotype in three representative populations. The subsequent bar graph shows expression levels of total and exogenous reprogramming factors (0: OCT4; S: SOX2; K: KLF4) of LGMD2D iPSCs and the relative HIDEMs; shown are the average data from four different patients (data showing values of each patient are available in FIG. 9). The curves illustrate proliferation of three different LGMD2D HIDEMs vs. primary human MABs (black line), whereas histograms show surface markers. Bottom panel (DIFFERENTIATION) shows MyoD-ER-mediated myogenic conversion of three different HIDEMs (left column) and fusion of a representative population (marked with GFP) with C2C12 myoblasts (scale bar: 250 μm). (D) Myogenic differentiation via tamoxifen-induced MyoD-ER nuclear translocation into genetically corrected LGMD2D HIDEMs. The map represents a scheme of the muscle-specific SGCA lentivector (details in FIG. 9C). Immunofluorescence panel shows SGCA expression only in a differentiated myotube (white arrow and inset; scale bar: 40 μm). Western blot confirms immunofluorescence, demonstrating SGCA restoration into genetically corrected and differentiated HIDEMs.

FIG. 6. Transplantation of iPSC-derived MABs in Sgca-null/scid/beige mice.

(A) GFP fluorescence 7 days after intramuscular injection into a ribialis anterior muscle of 10⁶ genetically corrected LGMD2D HIDEMs (scale bar: 1 mm). (B) Immunofluorescence staining of a section from the muscle shown in (A) demonstrating engraftment of laminA/C+ human nuclei. Bottom picture shows a magnification of the area inside the white box showing a cluster of myofibers containing donor nuclei (scale bar: 50 μm). (C) Immunofluorescence showing a cluster of SGCA+ fibers containing human myonuclei one month after intramuscular transplantation of LGMD2D HIDEMs (quantified in the bar graph; scale bar: 40 μm). Bottom pictures show the same cluster in serial section stained for β- and γ-sarcoglycan (SGCB and SGCG). (D) Intra-arterial transplantation. Left panel shows vessel-associated GFP positive cells 6 hours after injection in the femoral artery of LGMD2D HIDEMs (scale bar: 0.5 mm). The right hand immunofluorescence pictures depict human cells in-between myofibers (scale bar: 50 μm) and the lower one depicts a human cell outside CD31+ vessels 12 hours after delivery (scale bar: 50 μm). (E) The bar graph illustrates genomic quantitative real-time PCR analysis for human telomerase (DNA) to detect engraftment (fold increase) of either HIDEMs (right leg) or their relative pre-reprogramming cells (left leg) 24 hours after intra-arterial transplantation (*** P<0.0005; unpaired t-test). (F) Representative example of SGCA+ myofibers containing human nuclei one month after intra-arterial transplantation of genetically corrected LGMD2D HIDEMs (scale bar: 50 μm). (G) RT-PCR confirming SGCA expression one month after intramuscular and intra-arterial injection. (H) Stereoscopic pictures of GFP+ myofibers one month after intra-specific transplantation of murine iPSC-derived MAB-like cells (MIDEMs). (I) Transversal sections from the muscle in (H) showing large areas of GFP and Sgca positive myofibers. (J) Time to exhaustion in treadmill tests of treated Sgca-null/scid/beige (n=13; 10⁶ cells injected bilaterally in tibialis anterior, gastrocnemius and quadriceps muscles) mice vs. untreated dystrophic (n=8) and non-dystrophic (n=5) controls, showing functional amelioration of mice transplanted with MIDEMs (+53.1% treated Sgca-null/scid/beige vs. +28.6% untreated Sgca-null/scid/beige). Note that data are presented as average motor capacity relative to baseline performances measured until the day before transplantation (*P<0.05; **P<0.005; one-way ANOVA).

FIG. 7. Additional characterization of HIDEMs derived from healthy donors.

(A) The image contains a 6-well culture plate stained for AP containing stable (left column) and unstable (right column) colonies from three different iPSC lines. Only stable clones were amplified and utilized for derivation of HIDEMs. (B) Shown is a nude mouse subcutaneously injected with HeLa cells (and sacrificed after 3 months) serving as a positive control for the tumorigenic assay described in Materials and Methods. (C) Principal component analysis of gene expression profiles of HIDEMs and several other cell types (listed in the right hand panel) revealing that HIDEMs show an higher level of correlation with human MABs and mesenchymal stem cells (MSC), a good level of correlation with smooth muscle (SM) cells, endothelial cells (END) and fibroblasts (FIB), whereas exhibit a low level of correlation with neural progenitors, ESCs and iPSCs. (D) Immunofluorescence (Z-stacks merge plus deconvolution) of a co-culture assay (GFP-positive HIDEMs with GFP-negative C2C12) showing a GFP-positive hybrid myotube containing a human myonucleus (scale bar: 20 μm). (E) Vascular network formation assays. The pictures on the left show two examples of tubular formation in co-culture with HUVEC cells on Matrigel™ gel, with integrated GFP positive HIDEMs; dashed line and “L” indicate the lumen of the vessel. Top-right picture shows also the formation of similar structures by HIDEMs even without HUVEC cells on Matrigel™-coated dishes. The bar graph quantifies the number of GFP positive HIDEMs inside vascular/tubular structures. Upper scale bar: 40 μm; lower scale bar: 40 (F) Smooth muscle differentiation of HIDEMs upon TGF-β treatment for one week, demonstrated by α-smooth muscle actin expression; histrogram quantifying the extent of differentiation (scale bar: 40 μm). (G) FACS analysis of viral-integration-free HIDEMs. The panel also show enzymatic AP staining of the same cells (arrows), demonstrating higher sensitivity of the last technique (scale bar: 40 μm).

FIG. 8. Additional characterization of iPSCs derived from LGMD2D patients.

(A) Immunofluorescence analysis of pluripotency markers in LGMD2D iPSCs (scale bar: 140 μm). (B) The picture in the center shows 8 embyoid bodies, derived from the expansion of a single LGMD-2D iPSC colony, after 5 days in growth medium (scale bar: 1.5 mm). Bottom panels show differentiated cells outgrowth from the upper embryoid bodies seeded on Matrigel™-coated dishes for ten days: ectodermal differentiation is suggested by the appearance of elongated, neuronal-like cells in the bright field (live-imaging) and demonstrated by the presence of clusters of nestin-positive cells; mesodermal differentiation is suggested by fibroblastoid (left picture) and vascular-like network formation (right picture) in the live imaging pictures and demonstrated by the expression of α-smooth muscle actin (αSMA) from cells outgrowth from EBs; endodermal differentiation is suggested by the presence of acinar-like structures (asterisks; which might also be neural rosettes) and then demonstrated by the presence of SOX17-positive cells in culture (white scale bars: 0.5 mm; black scale bar: 90 μm).

FIG. 9. Additional characterization of HIDEMs derived from LGMD2D patients.

(A) Quantitative real-time PCR analysis (bar graph) of total and exogenous reprogramming factor transcripts in iPSCs and relative HIDEMs generated from 4 different LGMD2D patients. (B) TRAP assay performed on 3 different LGMD2D samples as described in FIG. 3E. F: pre-reprogramming fibroblasts or myoblasts; is iPSCs; H: HIDEMs. (C) Detailed map of the human muscle specific SGCA lentiviral vector. (D) RT-PCR analysis of SGCA and myogenic regulatory factors (MYOD and MYOGENIN) in 3 terminally differentiated MyoD-ER- and SGCA-transduced LGMD2D HIDEMs (H5V is an endothelial cell line shown as a negative control).

FIG. 10. Generation and characterization of HIDEMs from DMD and DMD(DYS-HAC) iPSCs.

(A) Morphology and alkaline phosphatase (AP) staining of DMD(DYS-HAC)iPSCs (scale bar: 2 mm). (B) Morphology of HIDEMs derived from the iPSCs in (A) (scale bar: 50 μm). (C) Representative AP staining of DMD(DYS-HAC)HIDEMs (scale bar: 40 μm). (D) Immunofluorescence for LaminA/C demonstrating complete human origin of DMD HIDEMs (scale bar: 80 μm). (E) Representative FACS analysis of DMD(DYS-HAC)HIDEMs. (F) Representative immunofluorescence panel of MyoD-ER infected, tamoxifen-treated, differentiated DMD(DYS-HAC)HIDEMs (5 days in differentiation medium; scale bar: 40 μm). (F) Expression of human dystrophin and myogenin in differentiated DMD and DMD(DYS-HAC) HIDEMs (note that human dystrophin is only present in the genetically corrected cells).

FIG. 11. Generation and characterization of Sgca-null/scid/beige mouse.

(A) Genotyping PCRs for scid and Sgca mutations, showing specific bands for both mutations in the 3^{rd} lane from the left. (B) The bar graph shows hemocytometric white blood cell counts in Sgca/Null/scid/beige versus Sgca and scid/beige mice, demonstrating leucopenia in the triple mutant mice (black bar); the dot plots show FACS profiles analyzing the presence of T and B cells with anti-CD3/CD45 (lower row) and anti B220/CD45 (upper row) antibodies respectively, showing marked immune-depression in Sgca-null/scid/beige mice (left column) comparable to scid/beige mice (middle column) as opposed to immunocompetent Sgca-null mice (right column). (C) Lower picture shows 8 months-old Sgca-null/scid/beige female with severe kyphosis. (D) Immunofluorescence on sections from control scid/beige and Sgca-null/scid/beige mice showing absence of Sgca staining at the sarcolemma of muscle fibers (scale bar: 100 μm). (E) Western blot analysis demonstrating complete absence of Sgca protein in 3 different Sgca-null/scid/beige mice. (F) Hematoxylin & eosin and Masson trichrome staining (fibrotic tissue is stained in blue, muscle fibers in red) comparing diaphragm and tibialis anterior muscle histopathology of 1 month and 8 months-old Sgca-null versus Sgca-null/scid/beige mice (scale bar: 170 μm). G) Creatine kinase levels in different 4 months-old scid/beige, Sgca-null and Sgca-null/scid/beige mice. (H) Survival curves comparing immunocompetent and immune-deficient Sgca-Null and scid/beige mice mortality. (I) Sgca-null/scid/beige mouse as a xenotransplantation recipient: injection of adult human MABs as a proof-of-principle. Top-left: immunofluorescence showing a cluster of Sgca positive fibers 3 weeks after intra-muscular transplantation of 5×10⁵ cells (scale bar: 50 μm). Top-center: the histograms show a quantitative real-time PCR analysis for GFP mRNA 6 hrs after intra-muscular and intra-arterial delivery of GFP positive human MABs, demonstrating engraftment also after intra-arterial transplantation. Top-right: immunofluorescence pictures showing one SGCA and human dystrophin positive fiber containing two human lamin A/C positive nuclei after intra-arterial transplantation of hMABs (scale bar: 15 μm). Bottom panel: immunofluorescence demonstrating presence (left picture) of CD68 positive macrophages in close proximity of human cells three weeks after their intra-muscular delivery in a 3-months-old Sgca-null/scid/beige mouse and absence of the same infiltration into 2-weeks old transplanted mice (right picture).

FIG. 12. Derivation of mesoangioblast-like cells from murine iPSCs (MIDEMs).

(A) AP staining of murine iPSC colonies (scale bar: 0.8 mm). (B) MIDEM morphology and AP staining (scale bars: 50 μm). (C) FACS analysis of MIDEMs. (D) Myogenic differentiation of tamoxifen-treated MyoD-ER-transduced MIDEMs (scale bar: 60 μm).

FIG. 13. Generation of human ES cell-derived MAB-like cells (HEDEMs)

(A) Morphology of Shef3 and Shef6 human ES cells in culture (scale bar: 1 mm). (B) Morphology of HEDEMs derived from the cells in (A)(scale bar: 0.15 mm). (C) FACS analysis of HEDEMs surface markers (Shef6 as a representative sample). (D) Immunofluorescence staining for myosin heavy chain (red) demonstrating in vitro skeletal muscle differentiation of Shef3 and Shef6 HEDEMs. The cells have been transduced with a MyoD-ER lentiviral vector and 4OH-tamoxifen was then administered for two days prior to 5 days in differentiation medium (DMEM+2% horse serum).

FIG. 14. Reduction of fibrosis, increased force of contraction and contribution to the progenitor pool upon transplantation of MIDEMs

(A) Quantification of fibrosis in trans-planted versus control mice showing a reduction in fibrosis in transplanted mice (n=3). *P<0.05, Student's t test. The two images show representative Masson trichrome staining of tibialis anterior muscles from transplanted and control Sgca-null/scid/beige mice (blue, fibrotic infiltrate). Scale bar, 250 mm. (B) Force measurements 4 months after MIDEM transplantation. (Left graph) Normalized tetanic force of isolated tibialis anterior muscles from intramuscular and intra-arterially transplanted mice together with control nontransplanted dystrophic and nondystrophic mice (n≧3 per group). (Right graph) Mean values of specific force for a population of single myofibers dissected from transplanted and nontransplanted gas-trocnemius muscles (together with the controls; n values above columns). The arrow indicates a representative picture of a GFP+ myofiber analyzed in the assay. Scale bar, 60 mm. Error bars represent means±SD. *P<0.05; ***P<0.0005, one-way ANOVA and Student-Newman-Keuls test. ns, not significant. (C) Cryosection of MIDEM-transplanted tibialis anterior muscle stained for CD31 (Pecam; brown, immunohistochemistry; to mark blood vessels) and AP (blue, enzymatic reaction). A serial section shows the presence of GFP+ myofibers and interstitial cells, some of which co-localize with the vessels marked as described above. Scale bar, 80 mm. The bar graph quantifies the total number of AP+ cells per section of tibialis anterior muscle of 8-month-old Sgca-null/scid/beige mice after 1M transplantation with MIDEMs. Error bars represent means±SEM. *P<0.05; **P<0.005, one-way ANOVA and Tukey's test.

### BRIEF DESCRIPTION OF THE SEQUENCES

SEQ ID NOS: 1-32 represent primers used in the Examples

### DETAILED DESCRIPTION OF THE INVENTION

Induced pluripotent stem cells (iPSCs) are the product of somatic cell reprogramming to an embryonic stem cell (ESC)-like state by specific transcription factors (1, 2). iPSCs extensively self-renew and generate differentiated progeny of all germ layers. The possibility of deriving patient-specific iPSCs to study diseases in vitro is a reality (3) and their genetic correction for autologous cell therapies is one of the most promising technologies for future personalized medicine (4). A critical step in designing iPSC-based protocols for skeletal muscle disorders is the development of techniques for their derivation and commitment into tissue-specific progenitors suitable for transplantation. Recent studies describe the generation of satellite cells (the main resident stem/progenitor cells involved in skeletal muscle regeneration) and their in vitro activated progeny (myoblasts) from murine iPSCs and from murine and human ES cells (5-7); however, these progenitors have the same limitations of bona-fide satellite cells for cell therapy purposes, i.e. no systemic delivery, poor survival and limited migration (8). Other mesoderm cell types have been shown to contribute to muscle regeneration, some of which (mostly PDGFRα+) can also be generated from mouse ES/iPS cell-derived embryoid bodies (EBs) (8-13). On the other hand, alkaline phosphatase-positive (AP+) human skeletal muscle pericytes, from which mesoangioblasts (MABs) are derived, represent one of the most promising cell populations, as they can be delivered systemically (14) and naturally contribute to skeletal muscle growth and regeneration (15). However, human MABs have a finite lifespan and the need to obtain billions of cells to treat all the skeletal muscles of a patient challenges their proliferative potency. Thus, the possibility of deriving MABs from PSCs offers the advantage of producing unlimited numbers of systemically deliverable myogenic progenitors.

Based upon pre-clinical evidence of safety and efficacy in dystrophic mice α-sarcoglycan-null and mdx, which model LGMD2D and DMD respectively) and Golden Retriever Muscular Dystrophy dogs (14, 16-22), a clinical trial of allogeneic MAB transplantation for Duchenne muscular dystrophy (DMD) is currently running in our Institution (EudraCT no. 2011-000176-33).

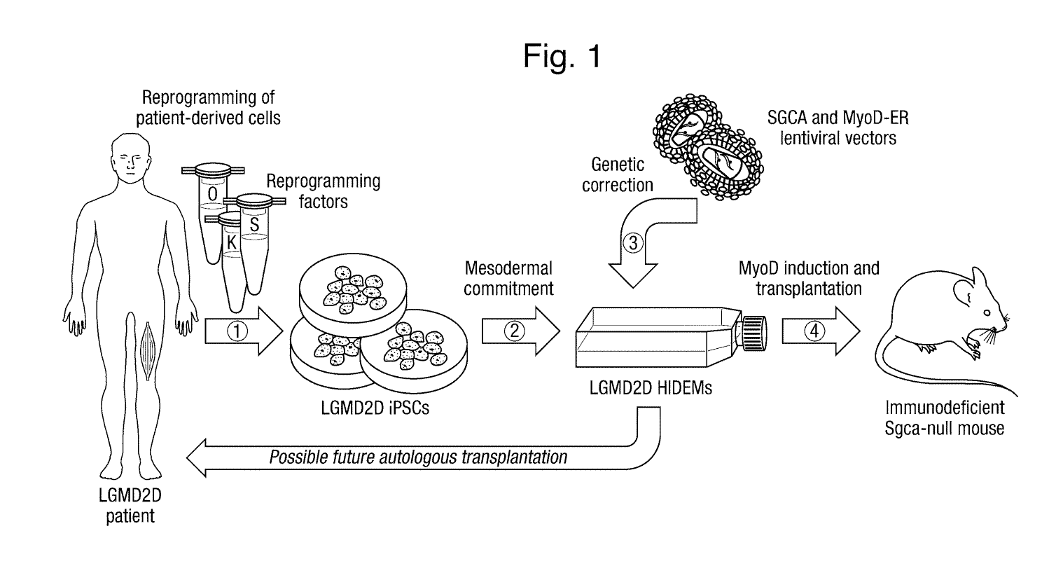

In order to develop an autologous cell therapy for LGMD2D (23, 24), we attempted to isolate human MABs from several patients, but invariably failed to derive cell populations with a MAB phenotype. Further analysis showed that these patients have a reduced number of AP+ pericytes in vivo. To overcome this problem, we developed a novel protocol to derive MAB-like cells (Human iPSC-Derived MABs: HIDEMs) initially from healthy iPSCs and subsequently from iPSCs reprogrammed from skeletal muscle cells of LGMD2D patients. HIDEMs can be easily expanded in culture, transduced with lentiviral vectors expressing human α-sarcoglycan (SGCA) and restore SGCA expression upon xenotransplantation (FIG. 1). Finally, we show functional amelioration upon intra-specific transplantation and extension of this strategy to other forms of muscular dystrophy and gene correction (i.e. DMD with human artificial chromosomes).

### Pluripotent Stem Cells

According to the invention, mesoangioblast (MAB)-like mesodermal stem/progenitor cells can be obtained from any suitable type of pluripotent stem cells. Induced pluripotent stem cells (iPSCs) are preferred. In some embodiments embryonic stem cells, including human or mouse embryonic stem cells, can also be used. In other embodiments, the pluripotent stem cells are not human embryonic stem cells. Preferably, the pluripotent stem cells of the invention are human cells.

### Cell Culture Substrates

The solid support used in methods of the invention is typically coated with a cell culture substrate. Any suitable substrate can be used. Preferred substrates include gelatinous mixtures of extracellular matrix proteins, such as Matrigel. Matrigel is the trade name for a gelatinous protein mixture secreted by Engelbreth-Holm-Swarm (EHS) mouse sarcoma cells and marketed by BD Biosciences and by Trevigen Inc under the name Cultrex BME. This mixture resembles the complex extracellular environment found in many tissues and is used by cell biologists as a substrate for cell culture.

### Dissociation of Colonies of Cells

Optionally, an inhibitor of cell colony formation may be added to improve survival of colonies after disassociation. One example is Rock inhibitor (see the Examples).

### Cell Seeding

Cell seeding is typically carried out in the presence of an appropriate culture medium. Appropriate cell density and temperature are also typically maintained. Culture media may be as defined in the section headed “Cell Cultures” (see below in the Examples). For example, Human MABs and HIDEMs can be cultured in MegaCell DMEM (Sigma, USA) as described (37). Alternatively, cells can be cultured in Iscove's Modified Dulbecco's Medium (IMDM; Sigma) containing 10% FBS, 2 mM glutamine, 0.1 mM (3-mercaptoethanol, 1% NEAA, 5 ng/ml human bFGF, 100 IU ml⁻¹ penicillin, 100 mg/ml⁻¹ streptomycin, 0.5 μM oleic and linoleic acids (Sigma), 1.5 μM Iron [II] cloride tetrahydrate (Fe⁺⁺; Sigma), 0.12 μM Iron [III] nitrate nonahydrate (Fe⁺⁺⁺; Sigma) and 1% Insulin/Transferrin/Selenium (Gibco). Instead of 1.5 μM Iron [II] cloride tetrahydrate (Fe⁺⁺; Sigma), 0.12 μM Iron [III] nitrate nonahydrate (Fe⁺⁺⁺; Sigma), Fer-In-Sol (Mead Johnson), 0.12 μM Fe⁺⁺⁺ (Iron [III] nitrate nonahydrate (Sigma) or Ferlixit (Aventis) can also be used.

After this point, other media, such as Alphamem, Alphamem then Megacell or an IMDM-based medium may be used.

### Cell Sorting

Cell sorting will typically be carried out using FACS.

### EXAMPLES

### Definitions

AP: alkaline phosphatase; DMD: Duchenne muscular dystrophy; EB: embryoid body; ESC: embryonic stem cell; HAC: human artificial chromosome; HIDEM: human iPSC-derived mesoangioblast; iPSC: induced pluripotent stem cell; LGMD2D: limb-girdle muscular dystrophy 2D; MAB: mesoangioblast; MEF: mouse embryonic fibroblast; MIDEM: murine IDEM; PSC: pluripotent stem cell; SGCA/B/C: α-/β-/γ-sarcoglycan.

### Example 1

### Characterisation, Generation and Use of Mab-Like Cells

LGMD2D Patients have a Reduced Number of Skeletal Muscle AP+ Pericytes.

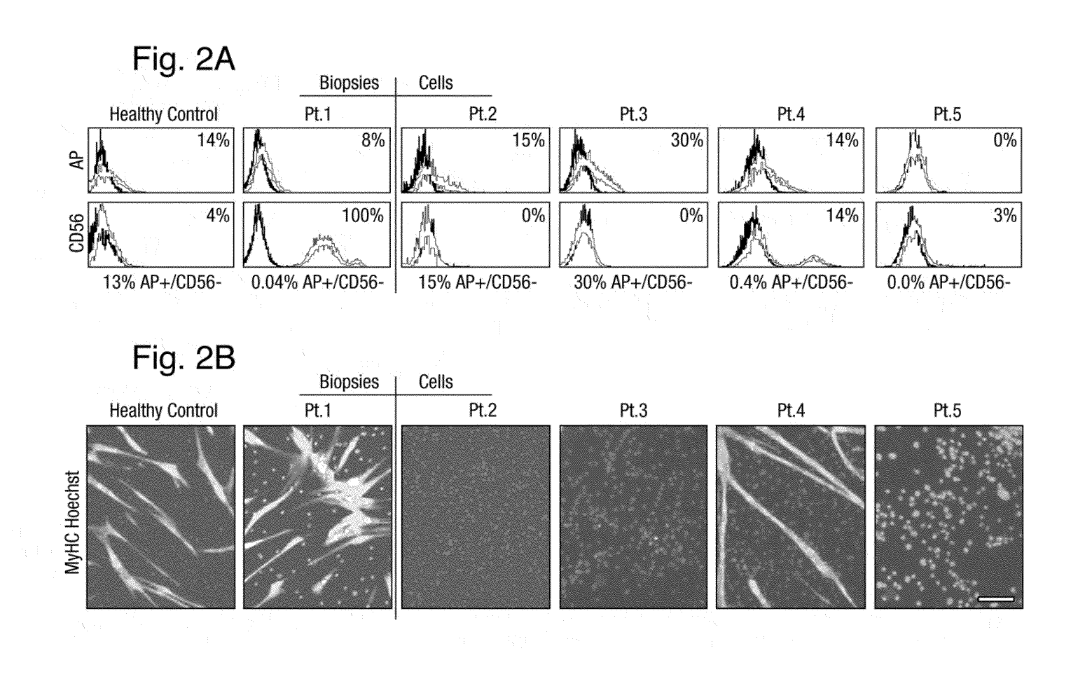

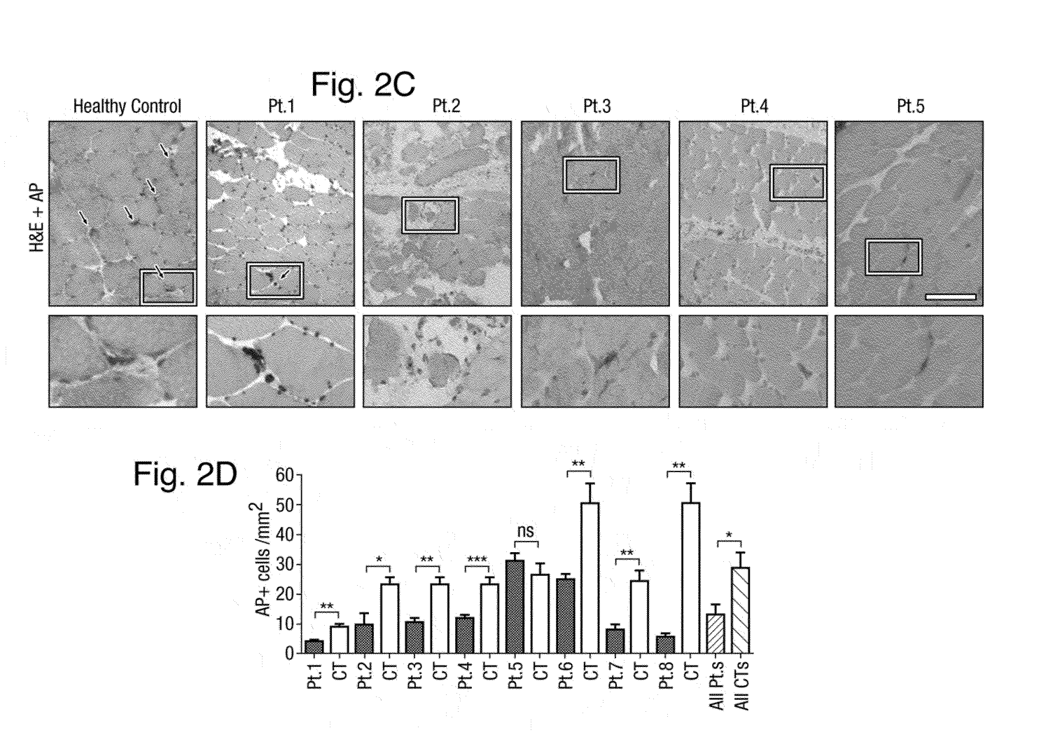

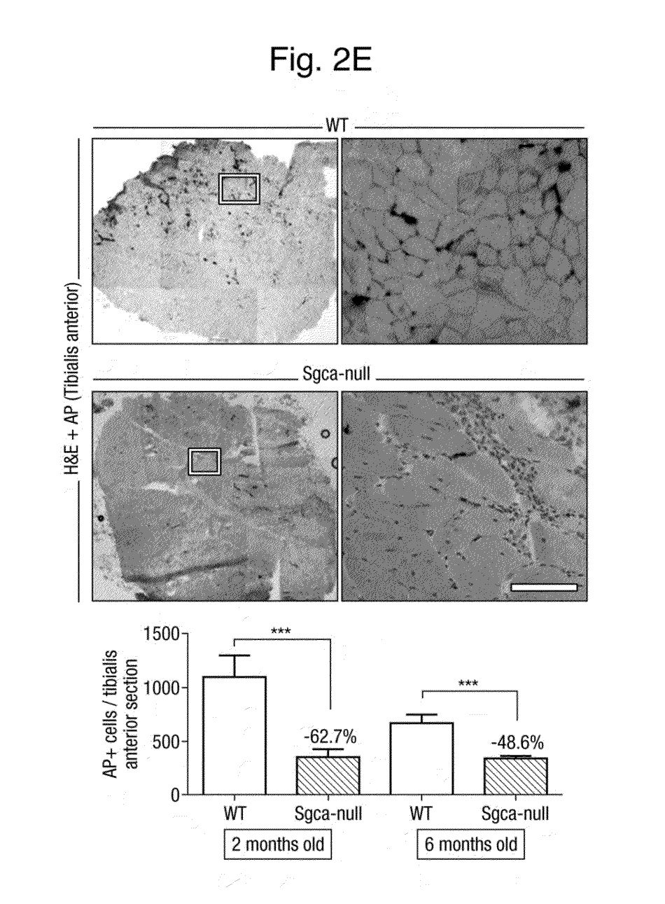

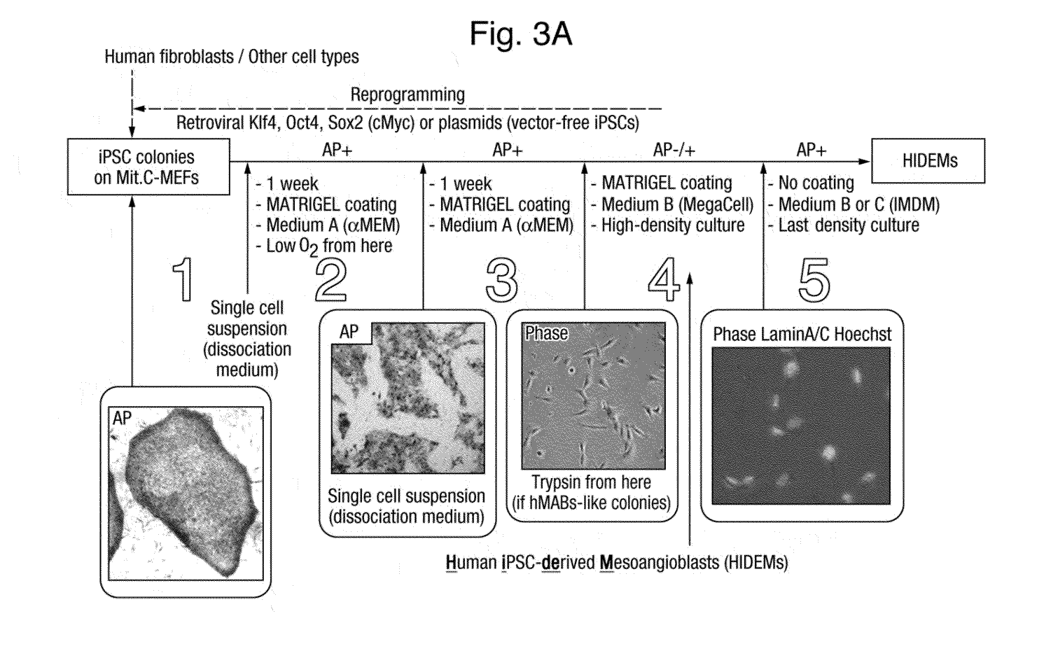

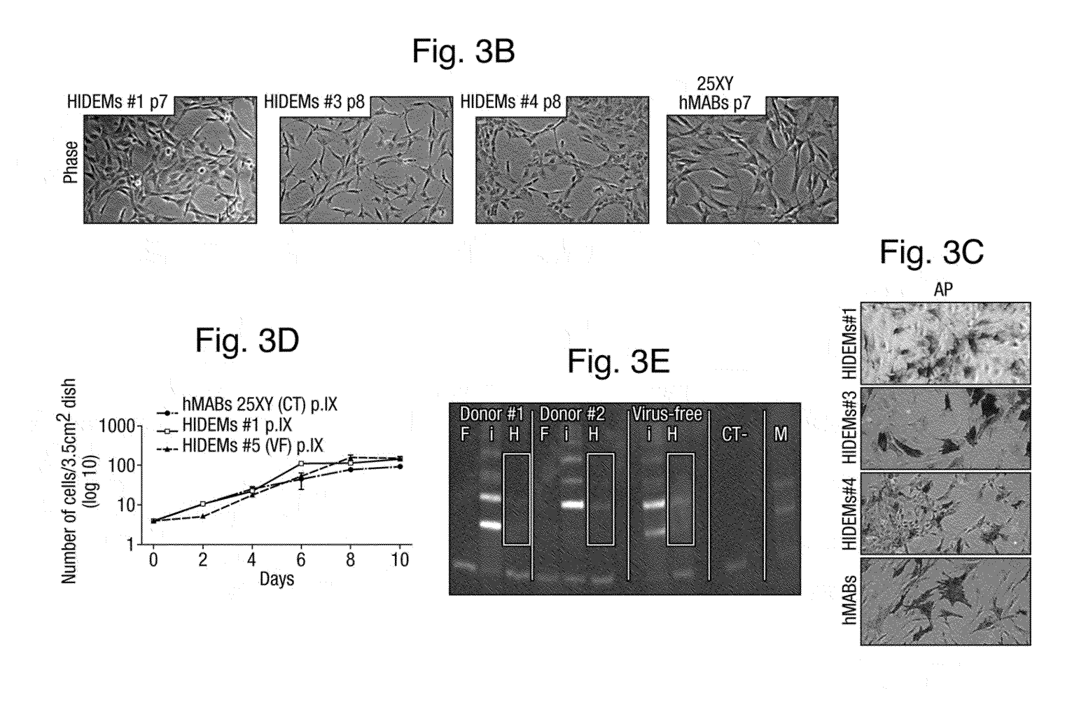

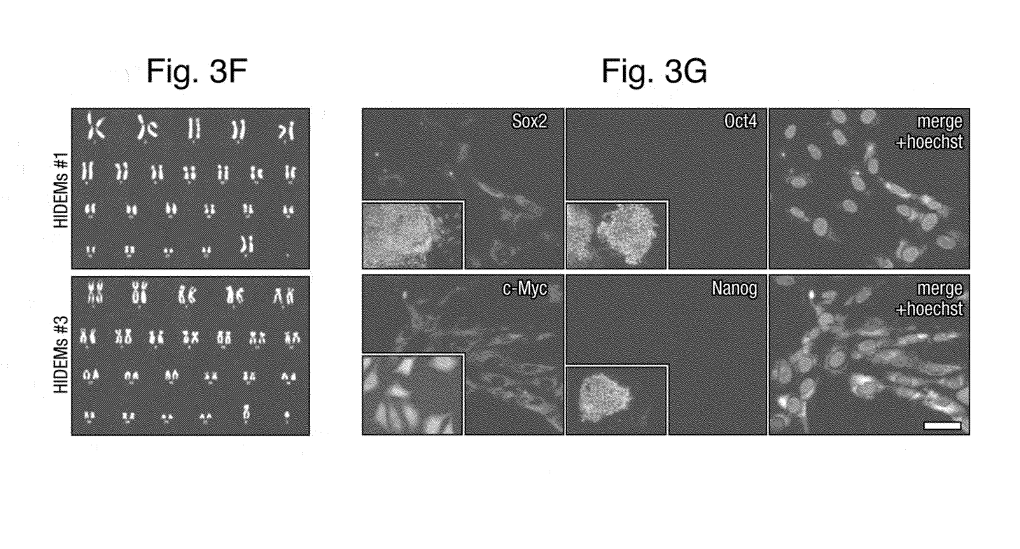

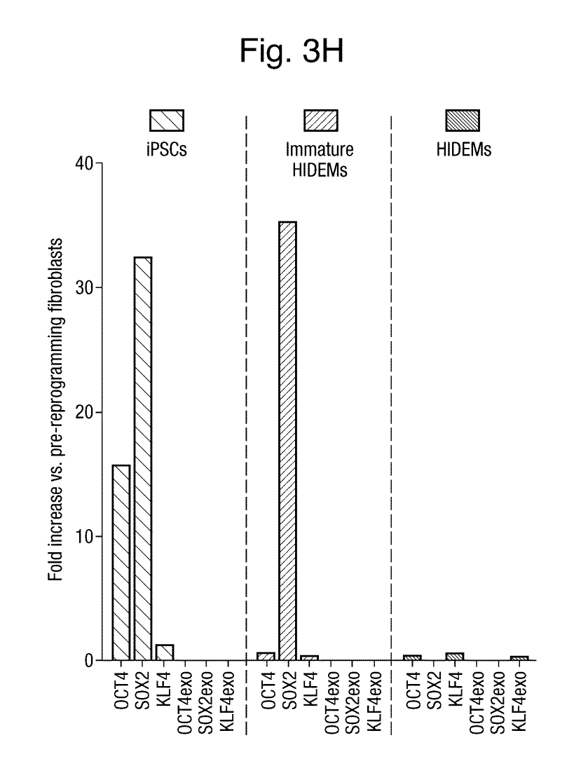

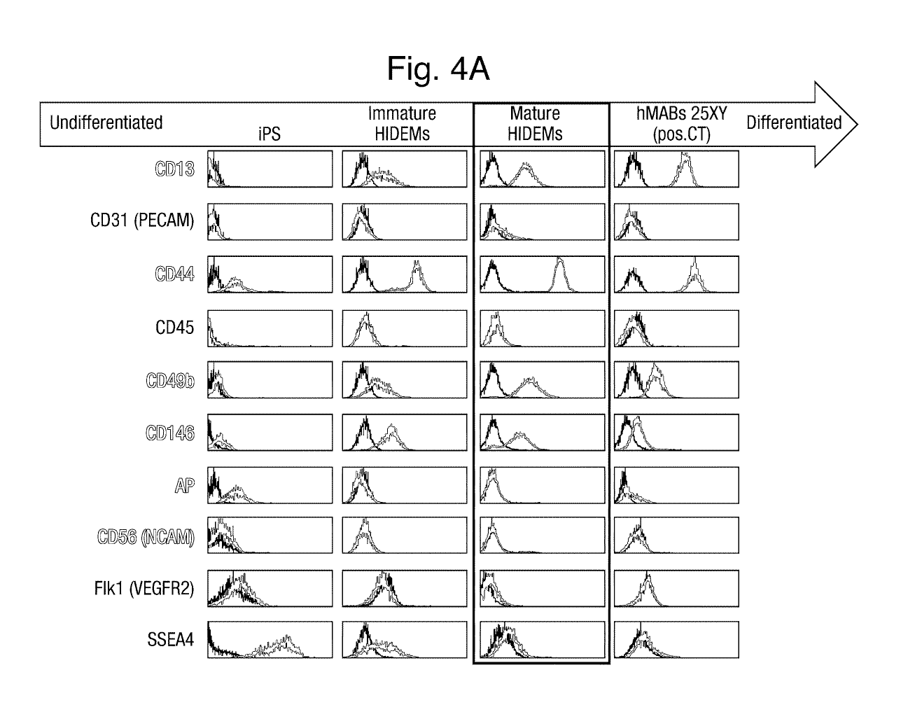

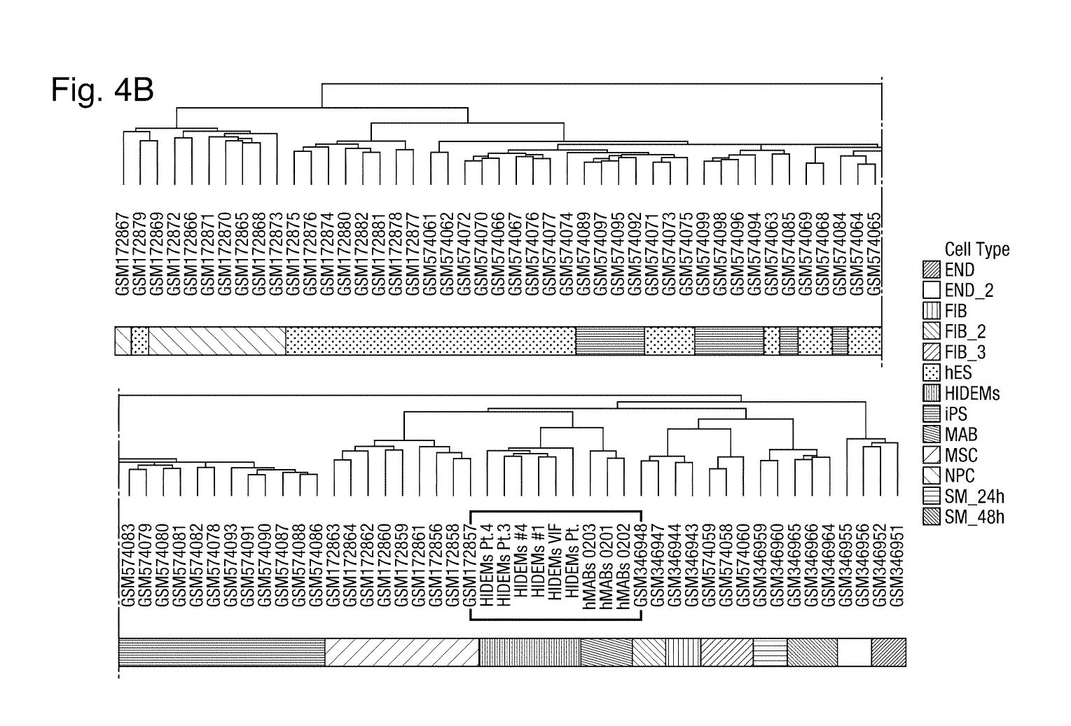

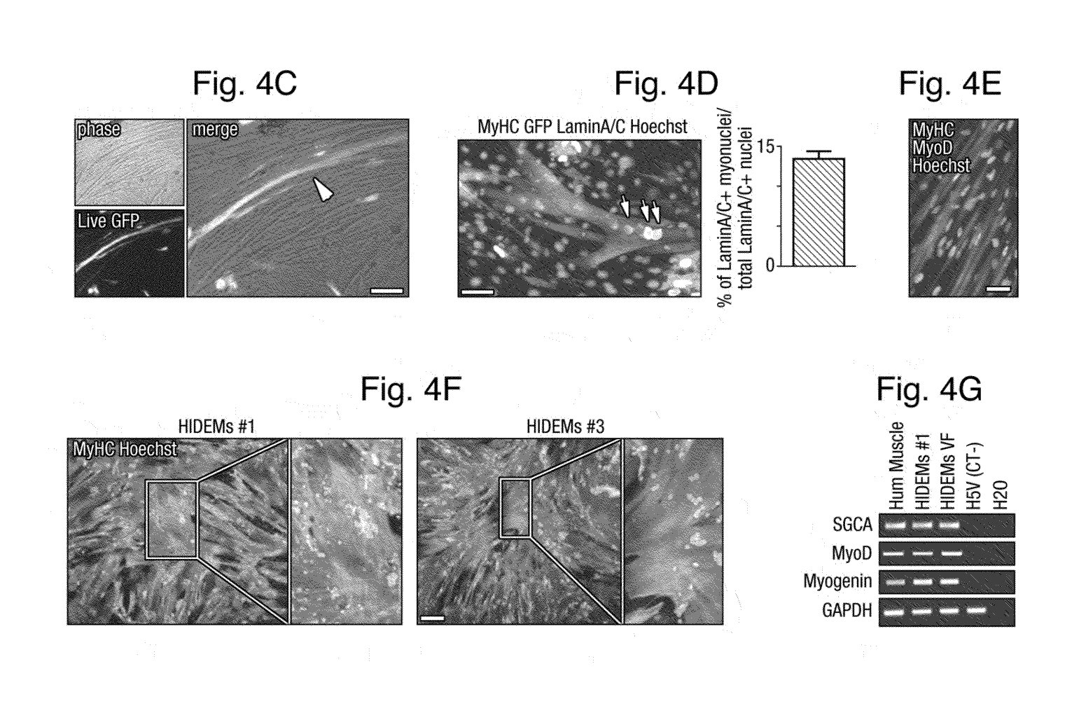

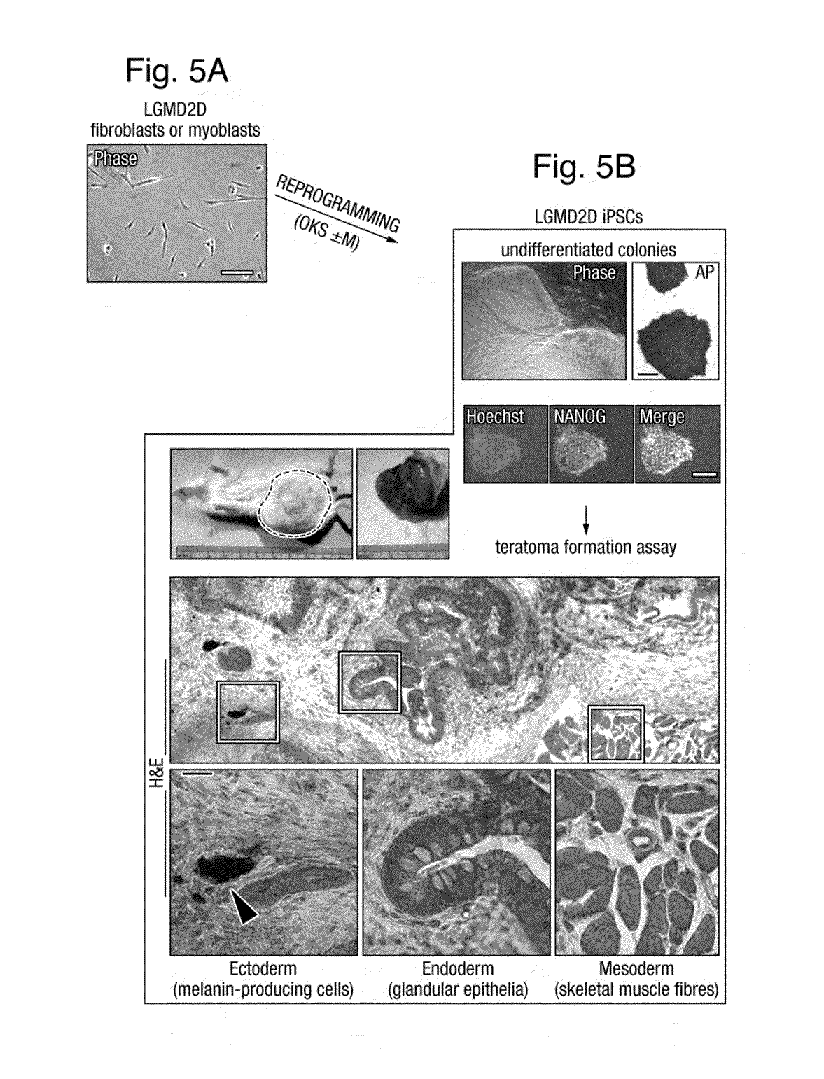

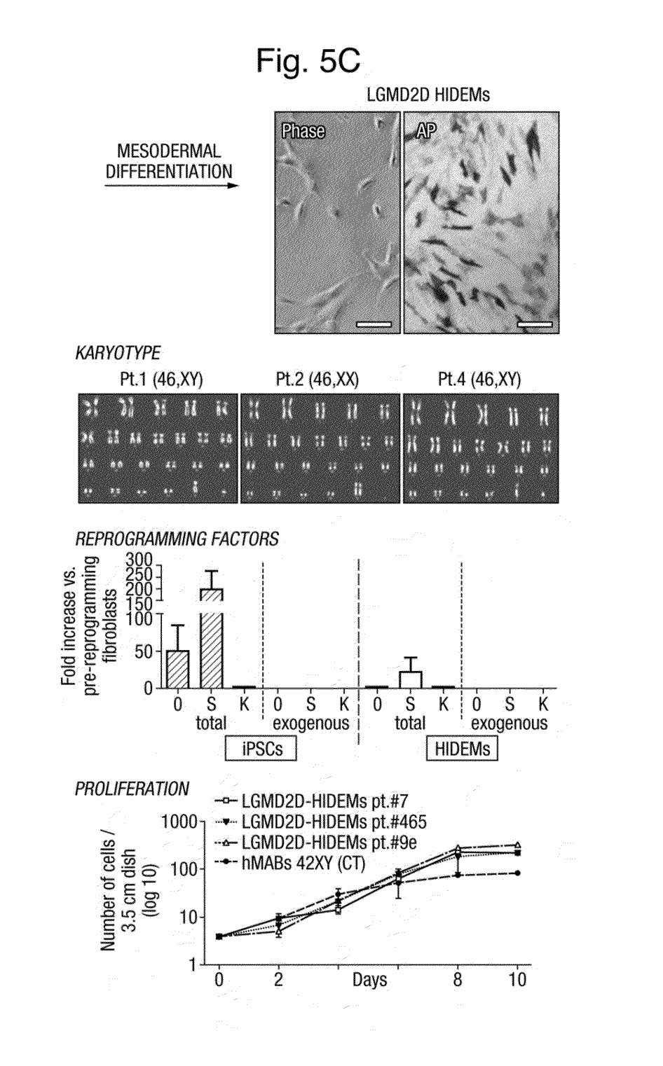

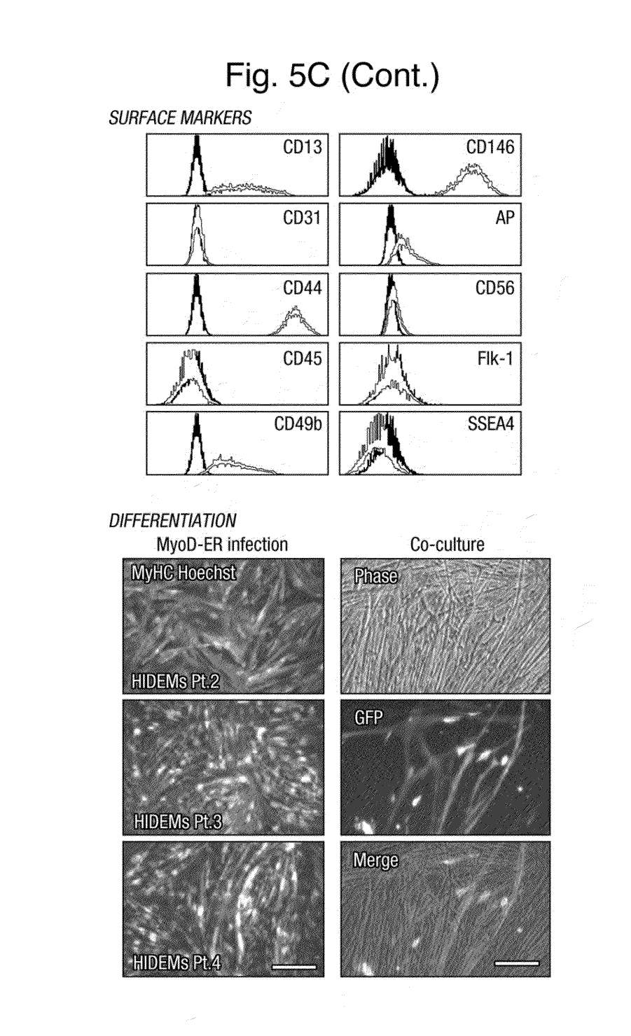

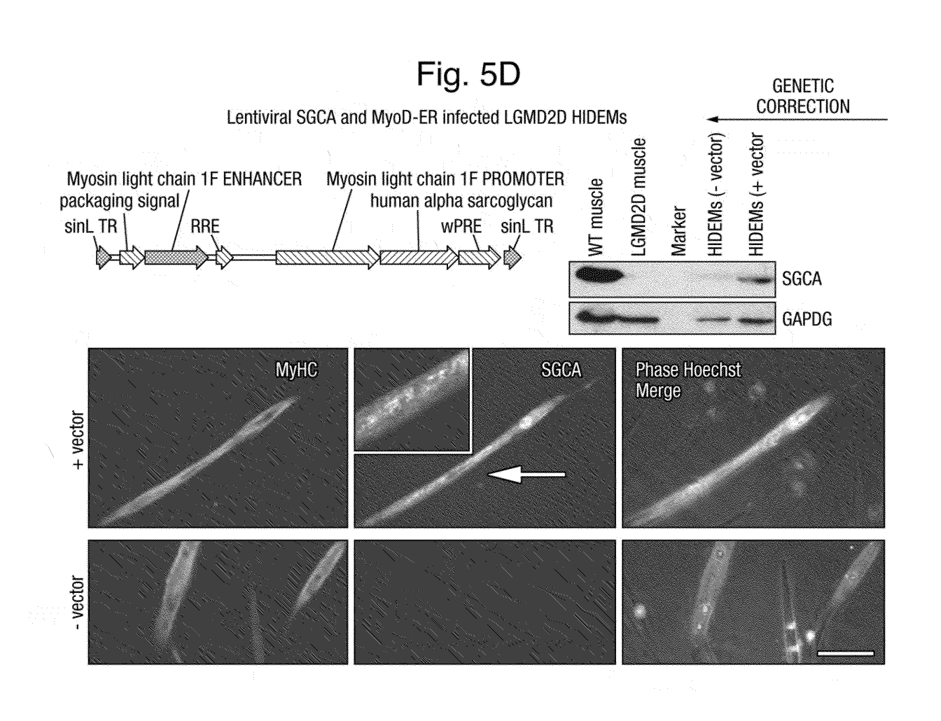

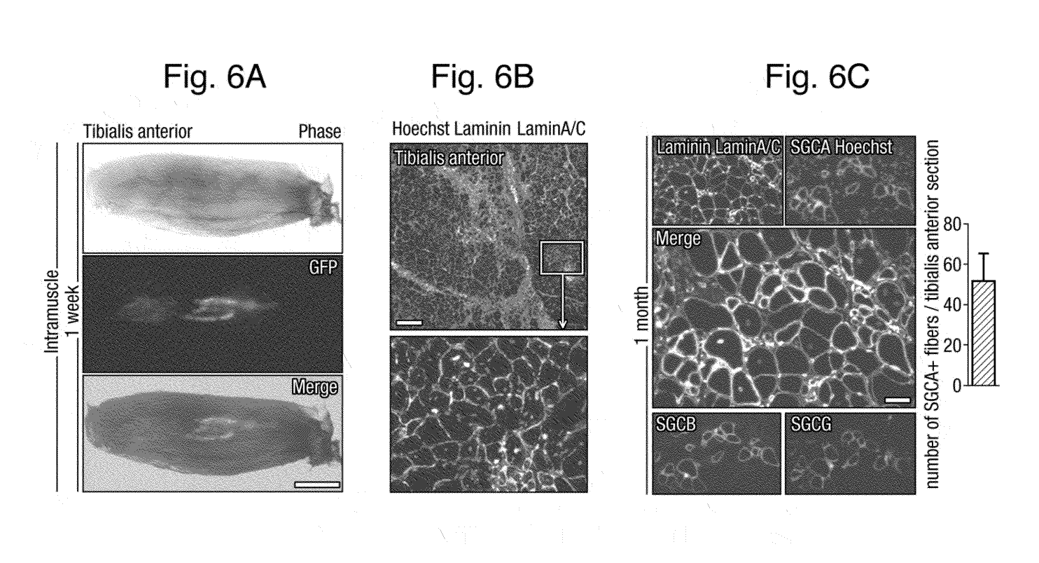

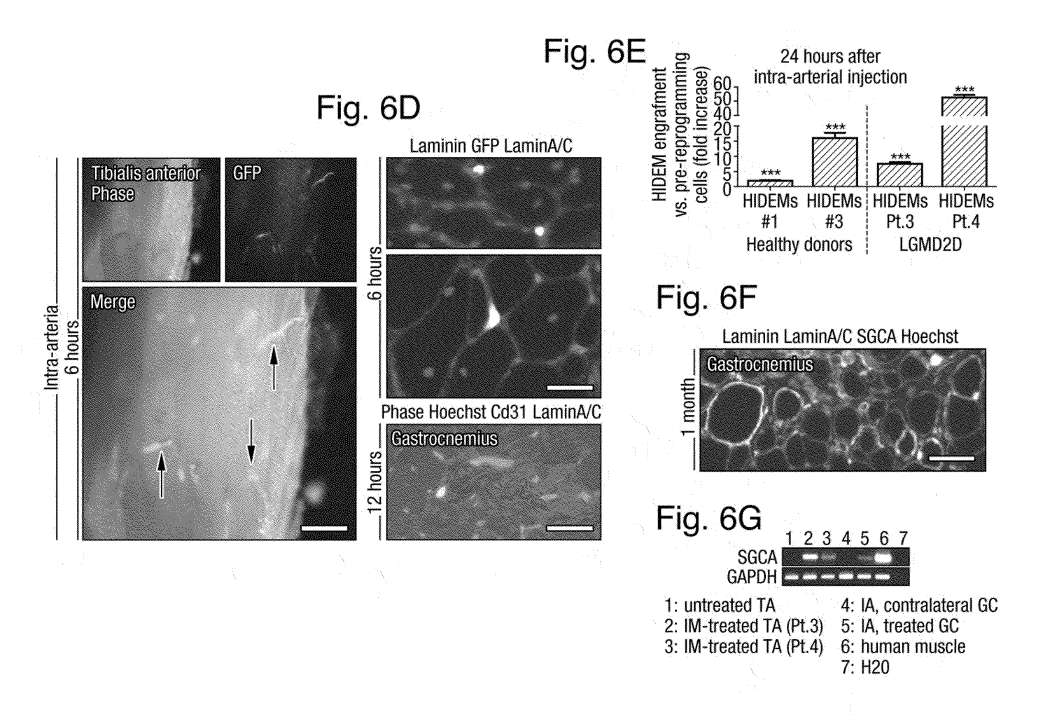

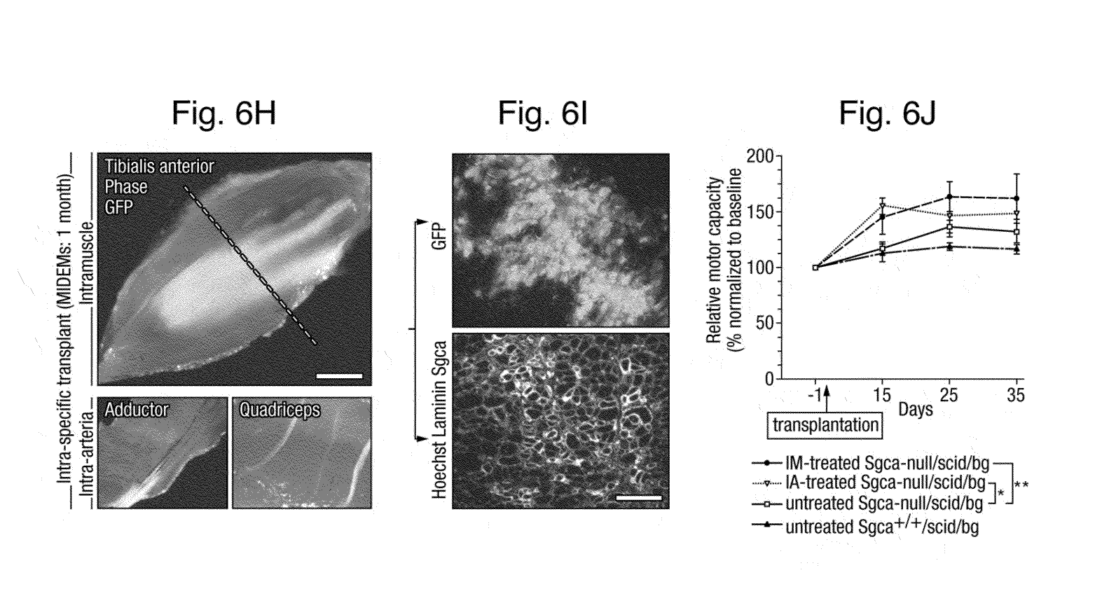

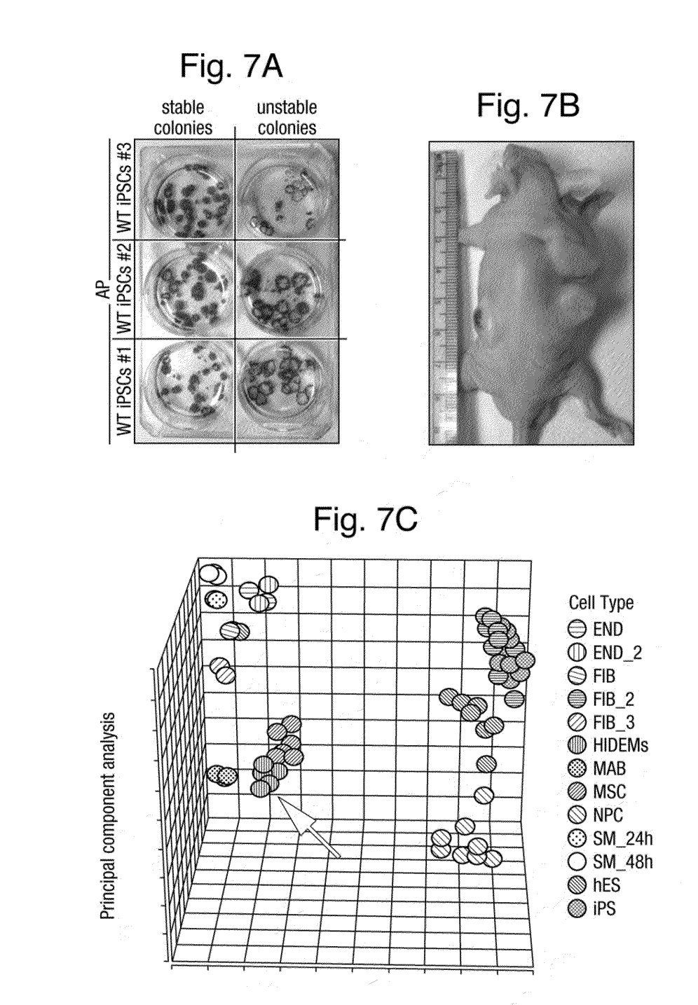

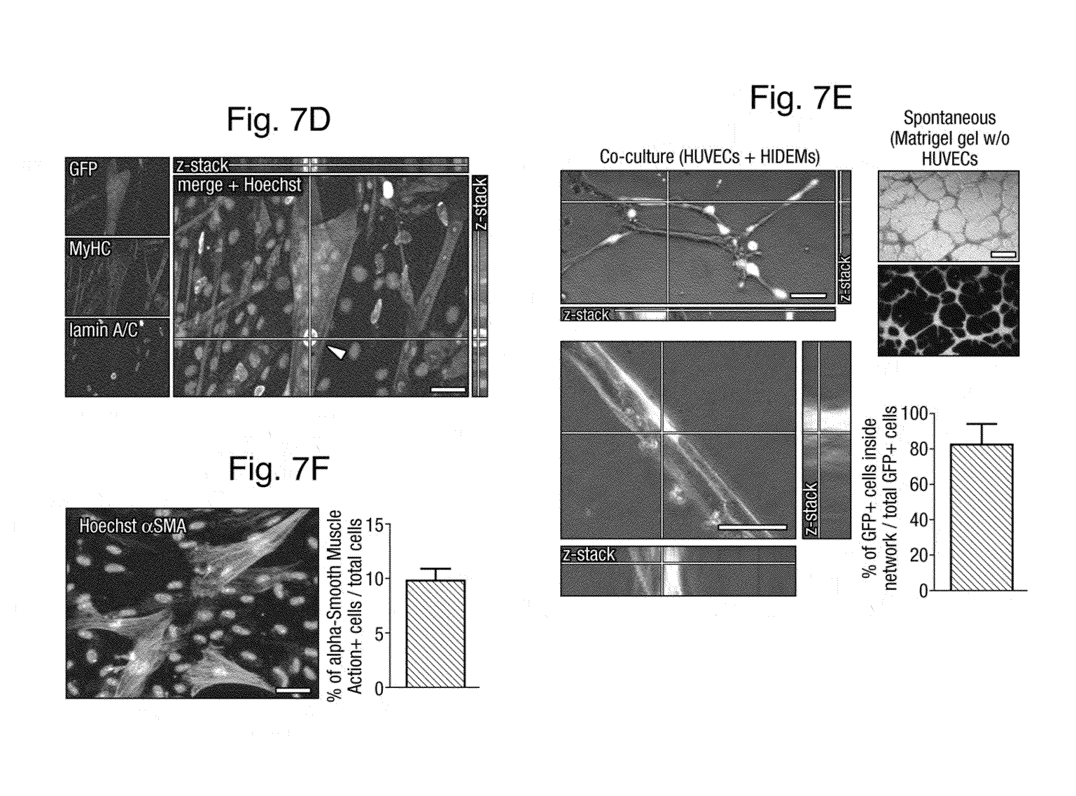

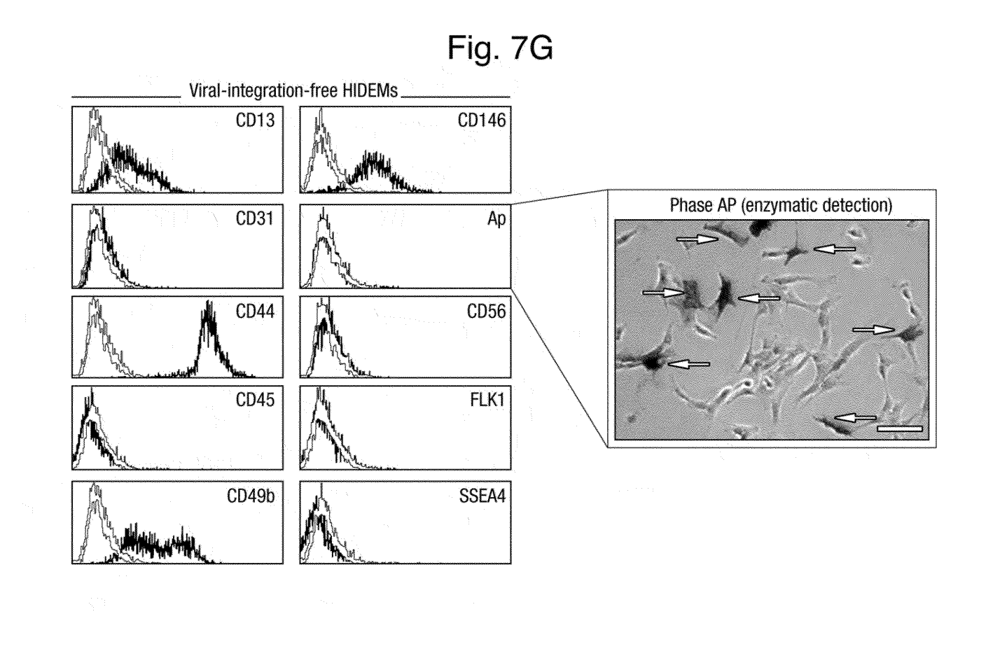

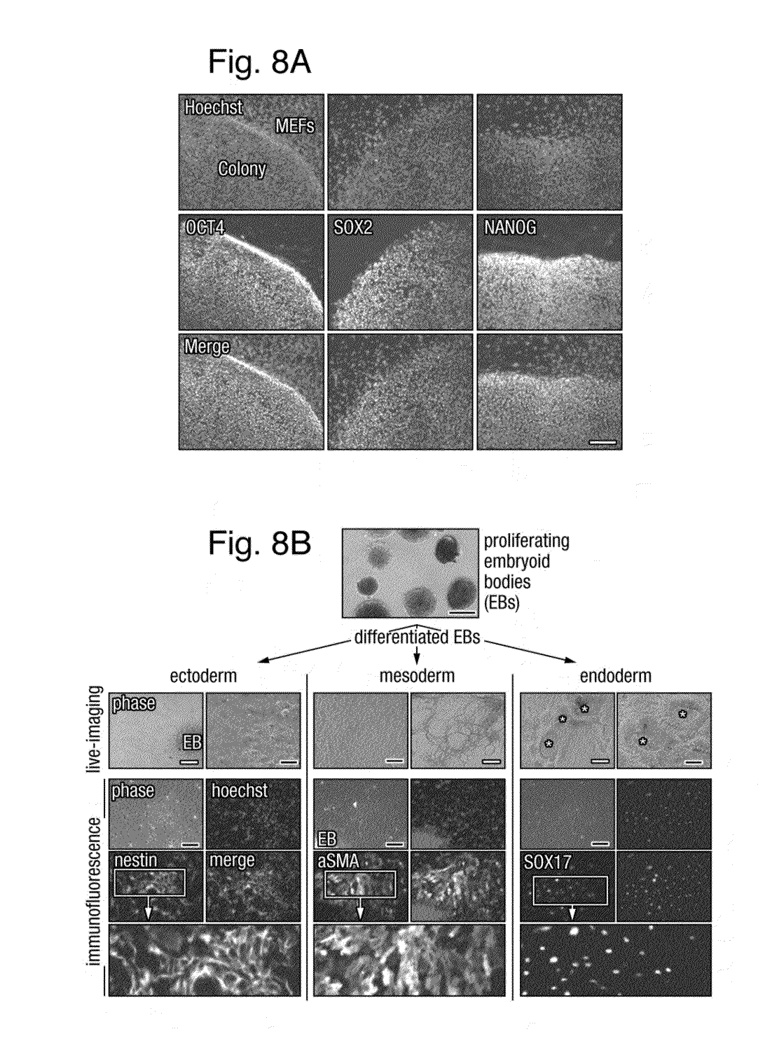

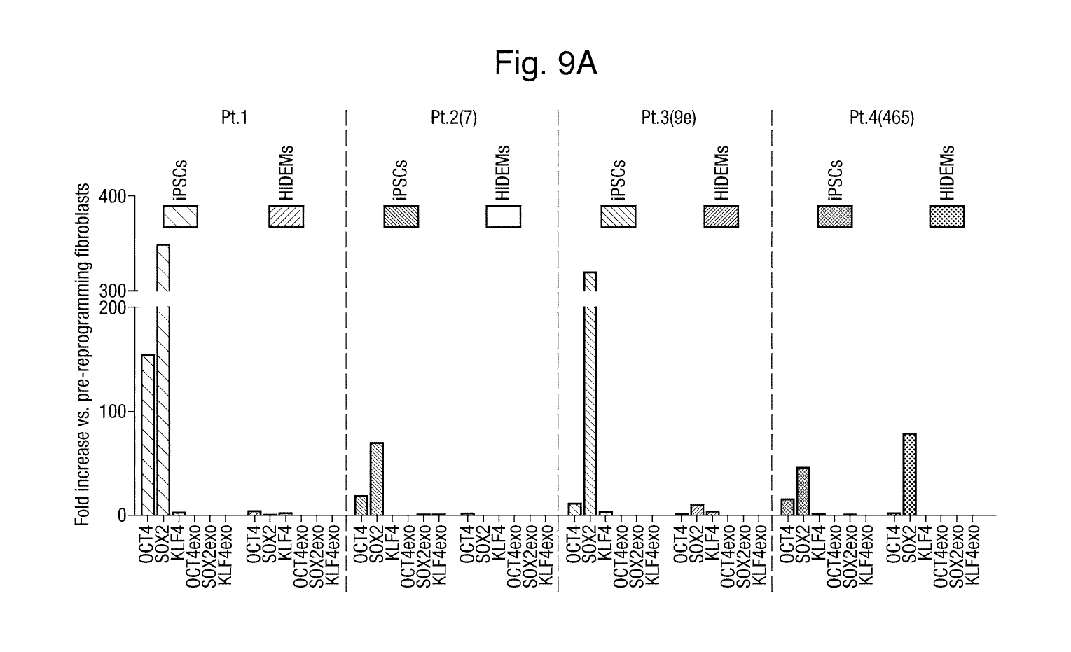

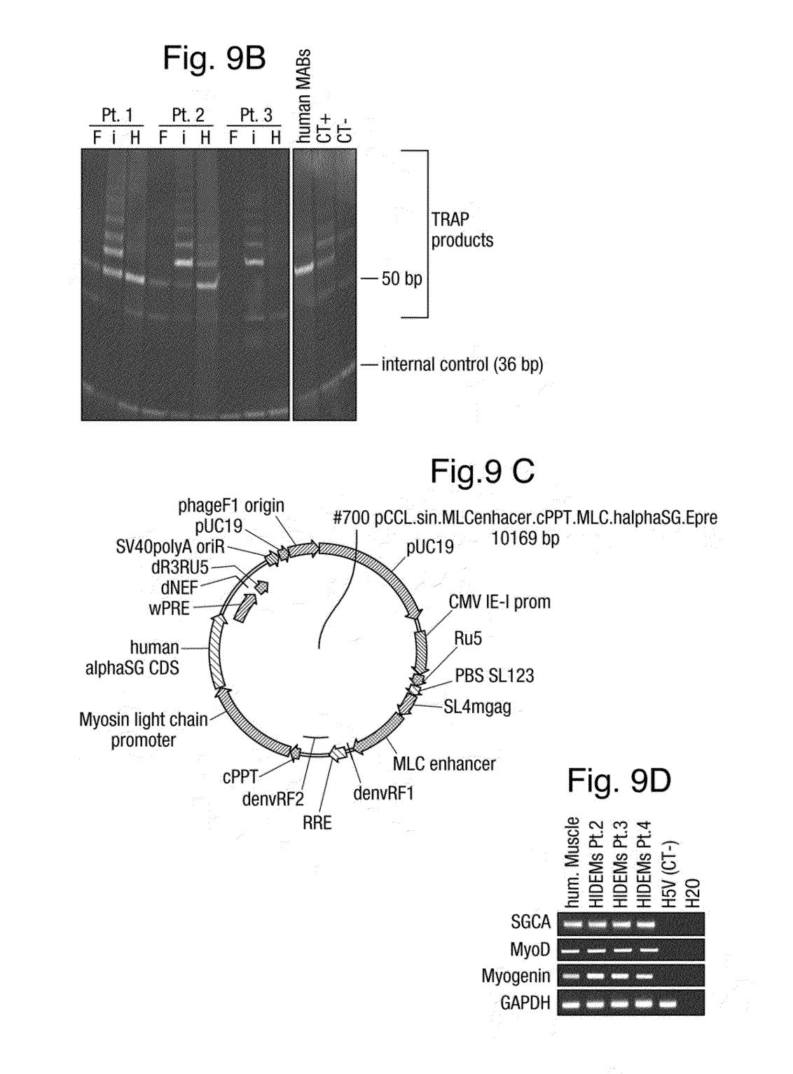

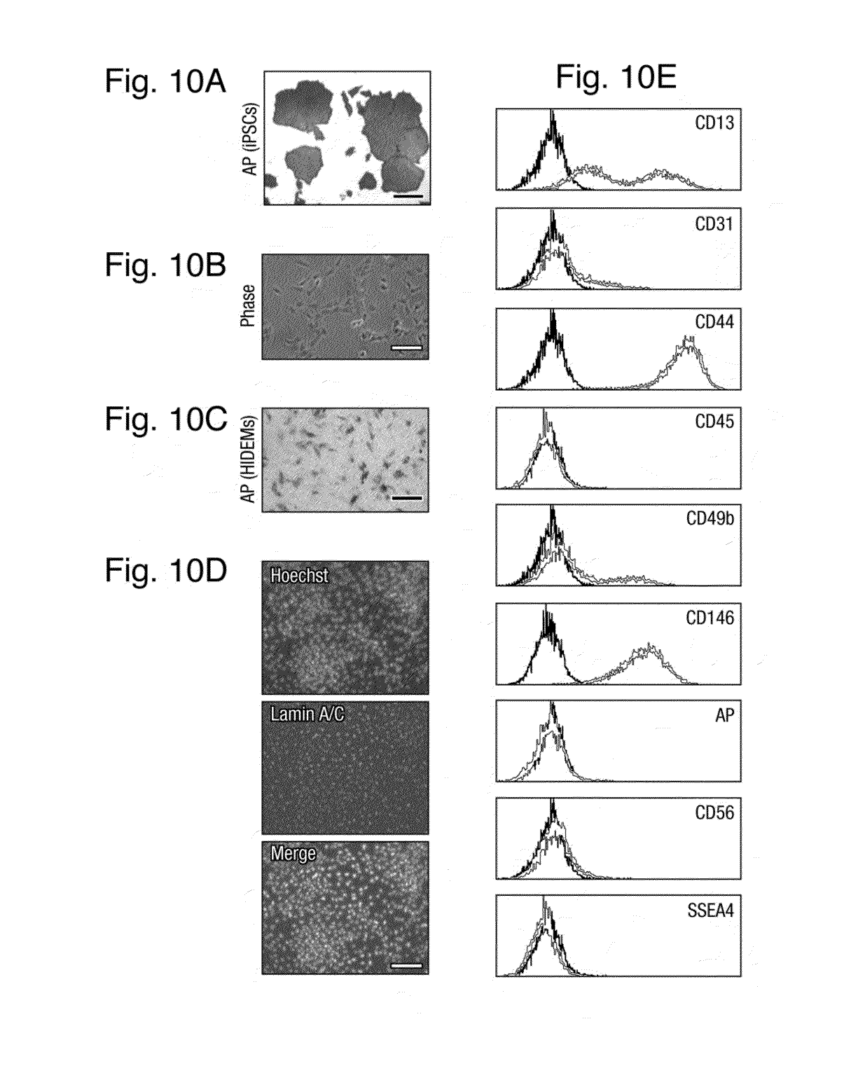

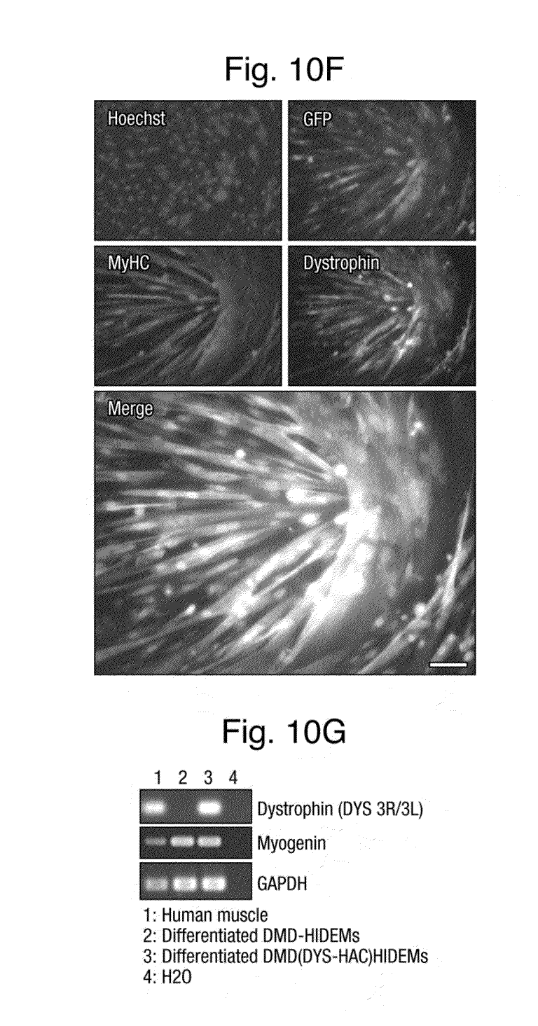

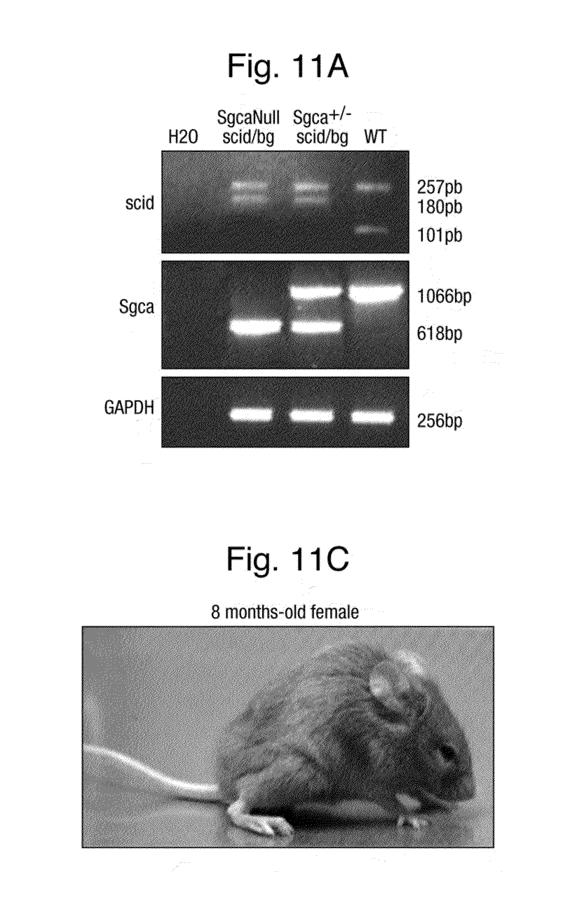

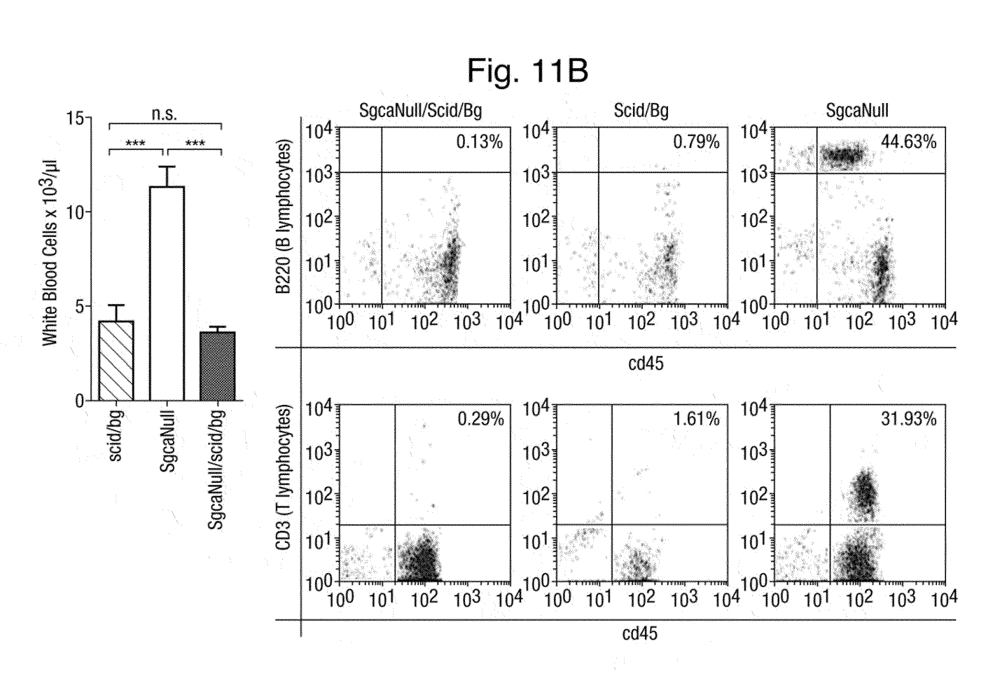

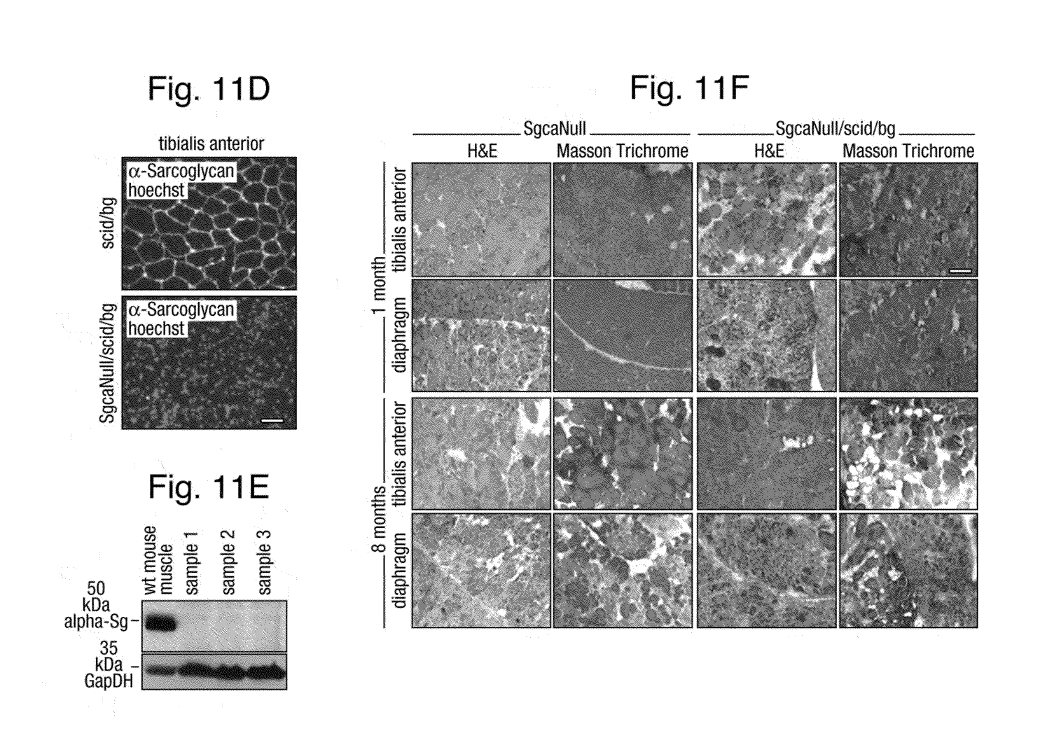

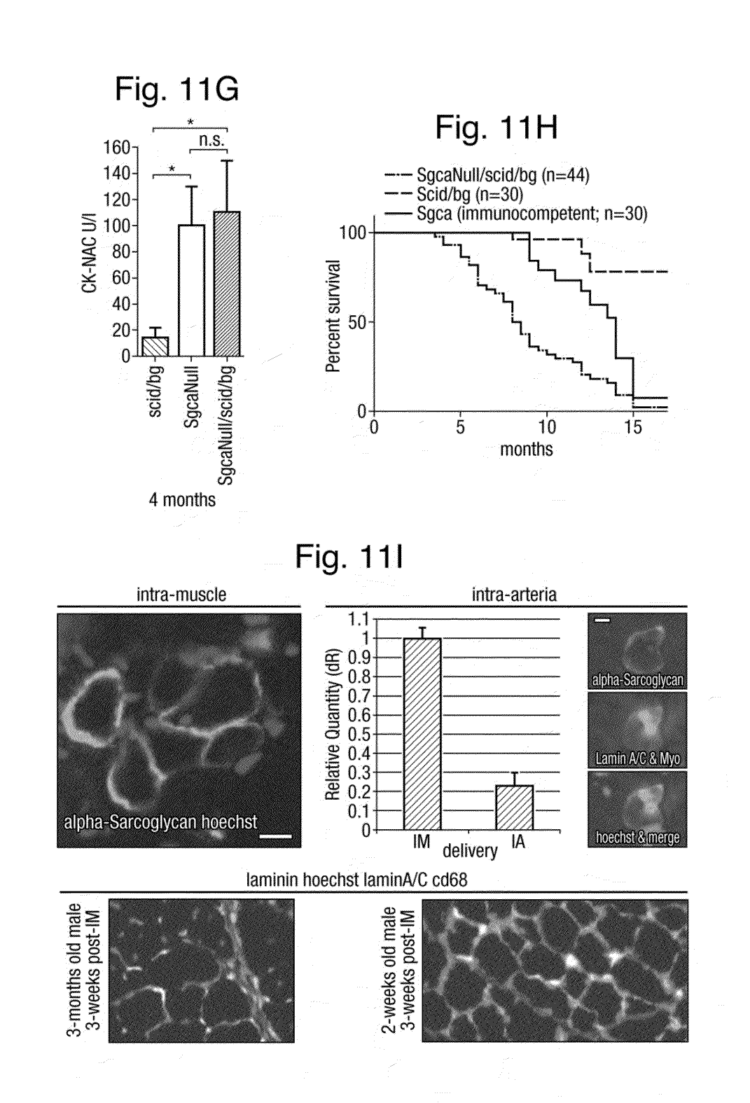

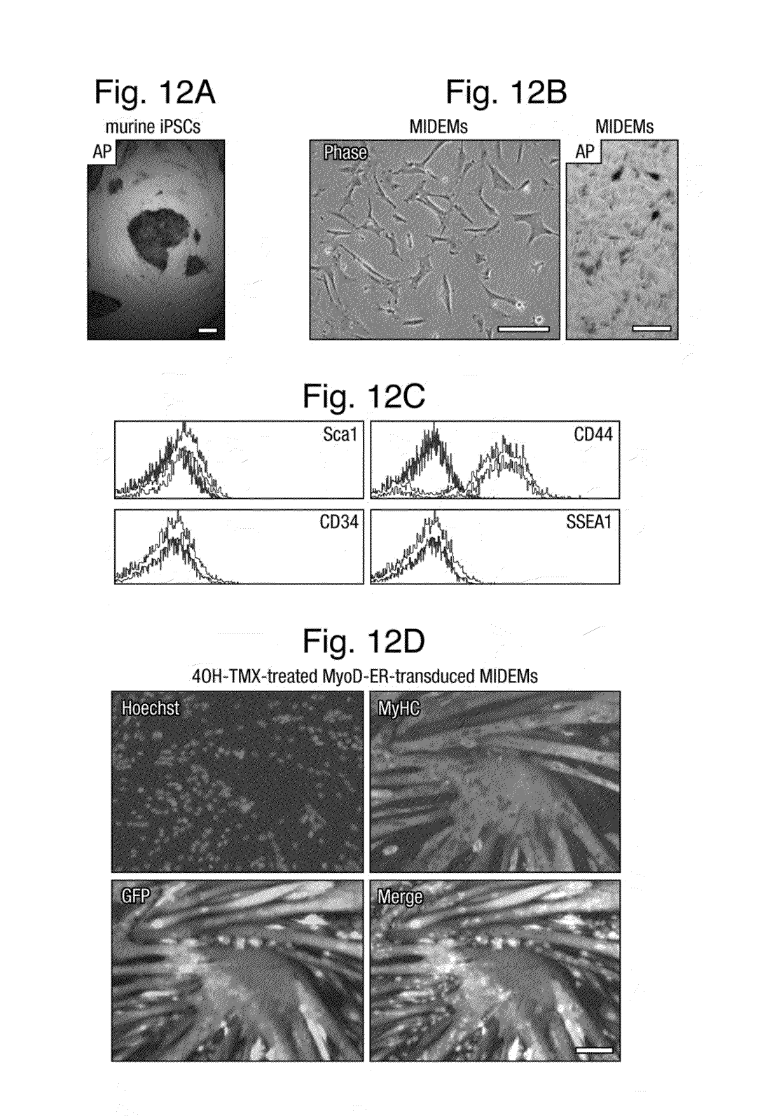

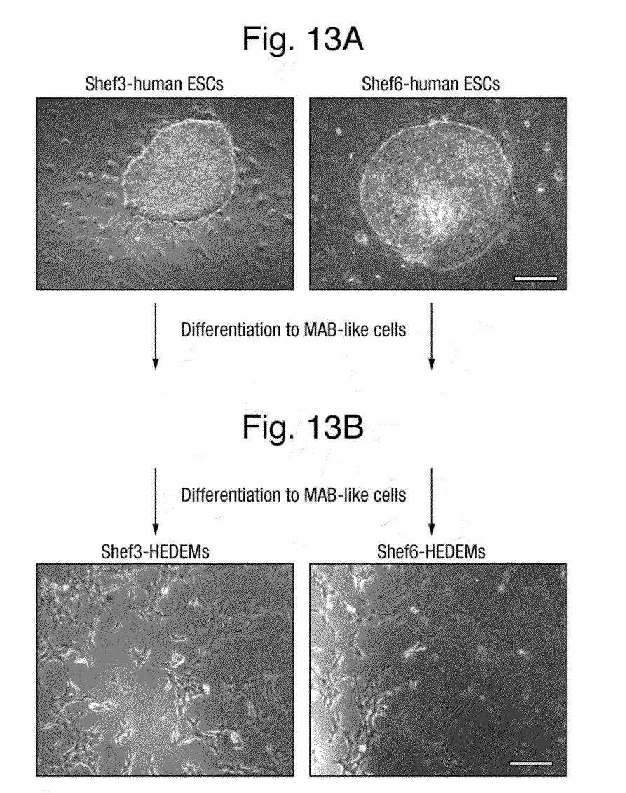

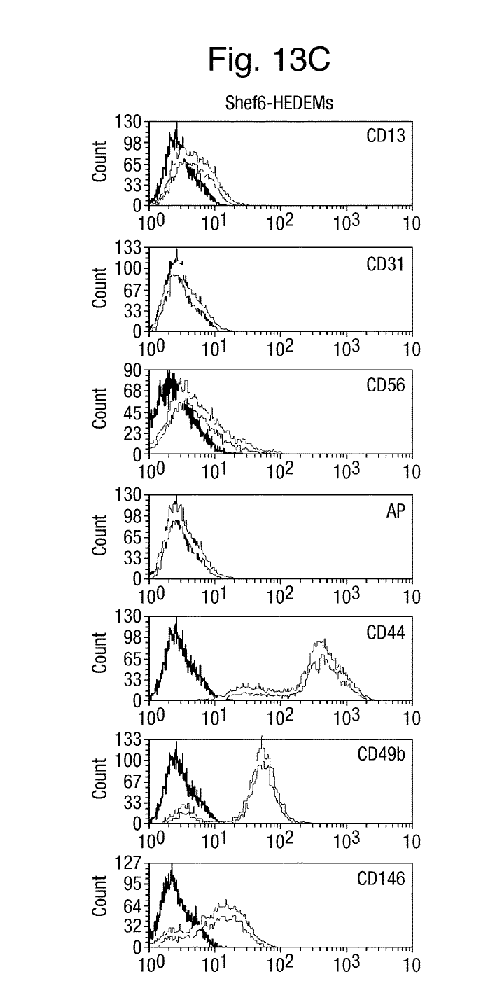

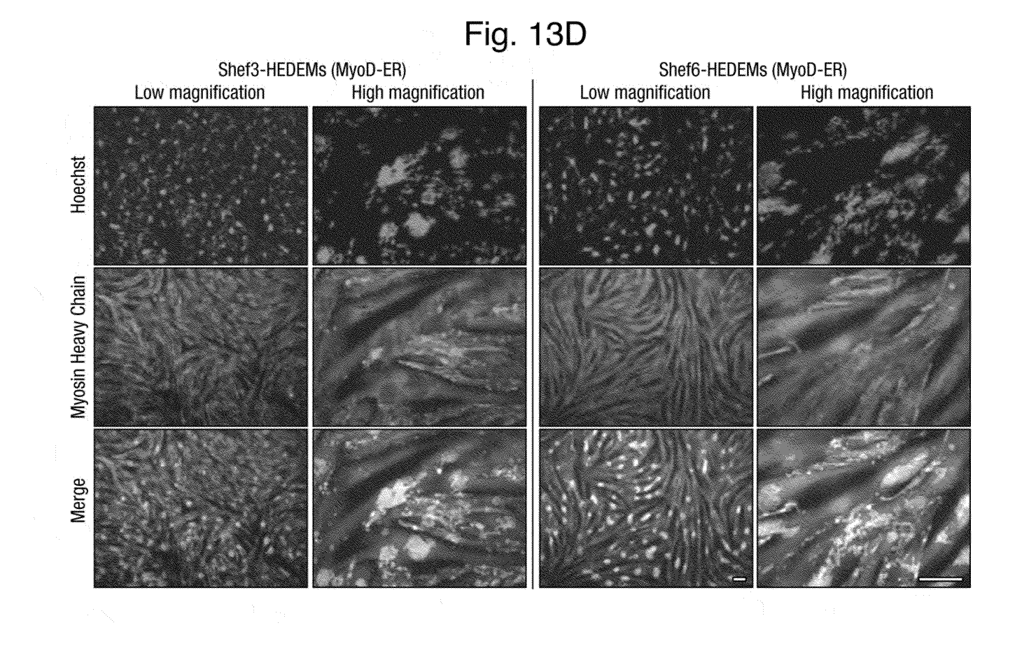

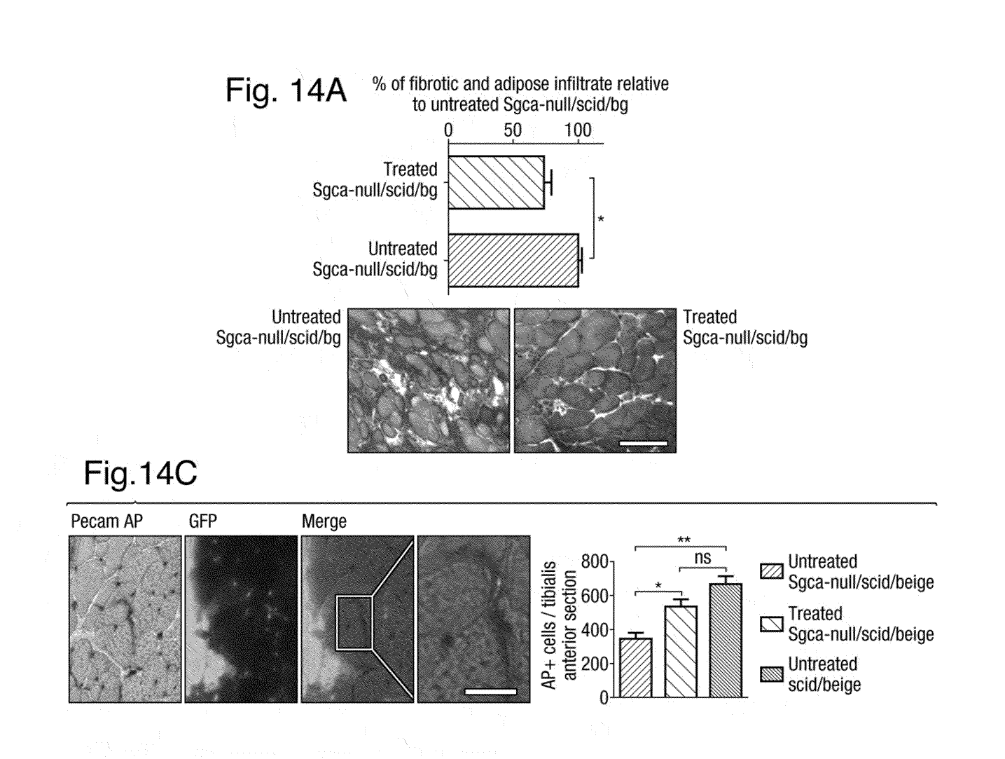

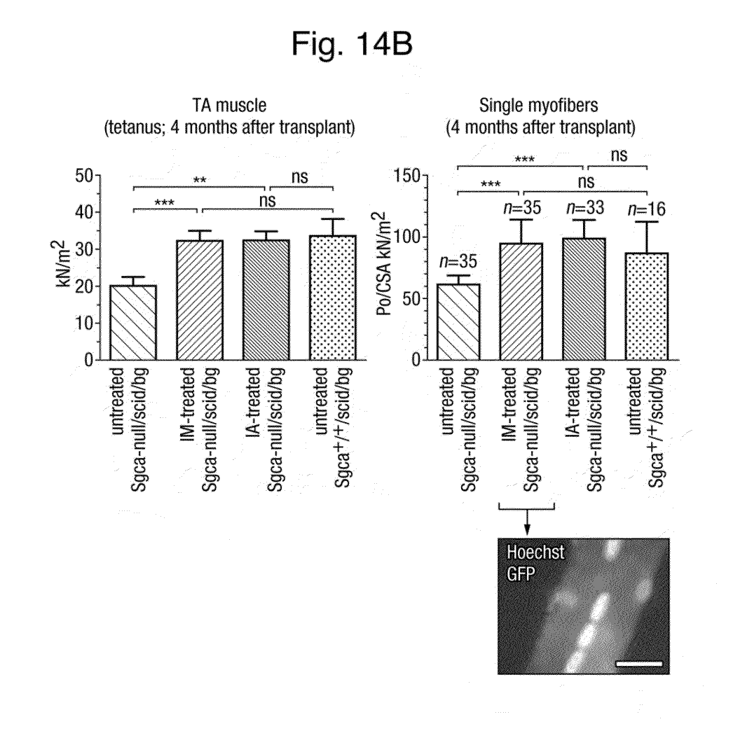

To test the therapeutic potential of human MABs for LGMD2D cell therapy, we first attempted to isolate them from LGMD2D muscular biopsies (table 1). Unfortunately, the isolation of AP+ pericyte-derived MABs was not successful, with the vast majority of outgrowing cells being CD56+ (NCAM1) myoblasts (FIG. 2A) that proliferated at a very low rate, as recently reported (25). Therefore, we tried to purify MABs from skeletal muscle cell preparations (from bio-banks; FIG. 2A and table 1) based upon expression of the markers AP and CD56: in four separate patients the few AP+ cells either did not proliferate in culture, or when they did, they were not able to undergo myogenic differentiation (Pt. 2, 3 and 5 in FIG. 2B). Furthermore, no cells at all outgrew from another bioptic sample (data not shown) and the last cell preparation (Pt. 5) contained mainly AP− and CD56− cells, presumably fibroblasts.

To explain this finding, we quantified the number of AP+ pericytes in sections from seven different LGMD2D skeletal muscle biopsies (five of which were obtained from the muscles utilized to generate the cells described above; see table 1). The results showed a strong reduction of AP+ cells in comparison with age-matched healthy controls (54.7%; FIG. 2C,D), suggesting a possible disease-specific cellular depletion/functional alteration of AP+ pericytes in LGMD2D. Notably, the same reduction of AP+ pericytes was observed in α-sarcoglycan-null (Sgca-null) (26) mice (FIG. 2E).

Generation of MAB-Like Stem/Progenitor Cells from Human iPSCs.

Among the possible strategies to overcome the limited availability of LGMD2D MABs, their derivation from PSCs appeared to be the most promising. To prove the feasibility of this strategy, we developed a method that allows easy, robust and relatively fast derivation (<3 weeks) of mesodermal stem/progenitor cells similar to MABs from healthy donor human iPSCs (human iPSC-derived mesoangioblast-like stem/progenitor cells: HIDEMs, details in Materials and Methods; detailed in FIG. 3A). Among other advantages, this protocol results in a homogeneous population of clonogenic (approximately 20% of colony forming efficiency, data not shown) and non-tumorigenic cells (0/27 immunodeficient mice), avoiding FACS-purification of EB-derived progeny.

HIDEMs resembled human MABs for morphology, AP expression and proliferation (FIG. 3B-E). Karyotype analysis demonstrated correct maintenance of ploidy into extensively passaged cells (>20 population doublings; FIG. 3F). Immunofluorescence and quantitative real-time PCR analyses revealed absence of reprogramming factors (FIG. 3G,H; details in the Supplementary Material (See Example 2)), with only one line having some residual SOX2-positive cells (data not shown), which did not interfere with differentiation, as recently reported (27).

Surface marker analysis (FIG. 4A) revealed up-regulation of MAB markers during the derivation process, in particular of CD13, CD44, CD49b and CD146 (an endothelial/perithelial marker) and a complete down-regulation of a pluripotency marker such as SSEA4. HIDEMs are CD56 negative, are negative or weakly-positive for endothelial markers (CD31 and Flk1), and are also variably positive for AP (after a transient down-regulation during the first differentiation steps; note that enzymatic reaction revealed AP presence also in FACS-negative samples; FIG. 7), like bona-fide human MABs (14). To compare HIDEM's molecular phenotype with those of MABs and other cell types (including PSCs), we first performed a gene expression profiling of HIDEMs (n=6) and human MAB (n=3) using the Affymetrix GeneChip technology, which revealed a striking similarity between the two populations. In addition, we downloaded from the GEO public repository 82 different datasets and performed meta-analysis using hierarchical clustering (FIG. 4B) and principal component analysis (FIG. 7 and Supplementary Material (See Example 2)). Both analyses revealed that gene expression profiles of HIDEMs show a very high level of correlation with MABs, a good level with mesoderm cells (mesenchymal stem cells, fibroblasts, smooth muscle and endothelial cells), whereas they exhibit a low level of correlation with neural progenitors, ES and iPS cells.

HIDEMs do not spontaneously differentiate into skeletal myocytes in vitro but, like embryonic MABs (28), their differentiation potential can be exploited by co-culture with myoblasts or by expression of the myogenic regulator MyoD (FIG. 4C-G). Indeed, upon transduction with a lentiviral vector containing a tamoxifen-inducible MyoD (MyoD-ER; see also Supplementary Material (See Example 2)) (29), HIDEMs underwent massive myogenic differentiation (FIG. 4F). Additionally, differentiation towards the vascular lineage was induced by TGF-β administration and vascular-like network formation was observed spontaneously and upon co-culture with human endothelial cells (FIG. 7E,F). Together these results demonstrate generation of a human mesoderm stem/progenitor cell type from iPSCs with MAB characteristics (see also FIG. 7).

Finally, we tested the possibility of deriving HIDEMs from certified viral-integration-free human iPSCs (see also Supplementary Material (See Example 2) for details): we robustly obtained cells with features comparable with the other HIDEMs, demonstrating that the presence of exogenous factors does not sustain their proliferative ability (FIGS. 3D, E and 7).

Reprogramming of LGMD2D Cells to iPSCs and Generation of Genetically Corrected HIDEMs.

After validation of the above protocol with healthy donor iPSCs, the cells obtained from the first four available patients (pt.s #1, 2, 3 and 4; representative example in FIG. 5A) were reprogrammed using retroviral vectors carrying SOX2, KLF4, OCT4±cMYC cDNAs (details in the Supplementary Material (See Example 2)). Colonies started to appear approximately 30 days after infection, with a global reprogramming efficiency at 45 days post-infection of 0.005% (using valproate and low O₂ culture conditions (30, 31)). Clonal lines were established from 4 different LGMD2D patients, with morphology comparable with human ESCs (FIG. 5B). Pluripotency was assessed by AP staining, expression of specific transcription factors, embryoid bodies and teratoma formation assay (FIGS. 5B and 8). Interestingly, we detected relatively low levels of KLF4 (FIG. 5C): this is in line with recent reports (32) and did not affect the pluripotency of our lines. Derivation and characterization of LGMD2D HIDEMs revealed that karyotype, proliferation, surface markers and myogenic differentiation were comparable with that of HIDEMs derived from healthy controls (FIGS. 5C and 9). Importantly, no reactivation of the exogenous transgenes was observed, even though endogenous SOX2 expression remained high in cells from one patient (specific levels in FIG. 9). No tumors developed in tumorigenic assays (0/36 immunodeficient mice).

In order to genetically correct LGMD2D HIDEMs, we developed a new lentiviral vector carrying the human α-sarcoglycan cDNA (SGCA) under the transcriptional control of the muscle-specific myosin light chain 1F promoter and enhancer (FIG. 9C). As shown in FIG. 5D, the transgene is selectively expressed in myotubes from genetically-corrected LGMD2D HIDEMs, previously transduced with the MyoD-ER lentivector (as opposed to surrounding cells not already differentiated). These data show that it is possible to reprogram LGMD2D cells to pluripotency, to genetically correct HIDEMs derived from LGMD2D iPSCs and that the resulting cells undergo terminal myogenic differentiation with correct and specific expression of the therapeutic transgene.

Additionally, we have also derived HIDEMs from DMD-iPSCs genetically corrected with a human artificial chromosome containing the entire dystrophin locus (DYS-HAC; FIG. 10) (33). We have recently shown efficacy of combined MAB transplantation and DYS-HAC-mediated genetic correction (19): this approach may open new therapeutic possibilities for this incurable myopathy, particularly in cases where primary human MAB derivation is impossible.

Transplantation of iPSC-Derived MAB-Like Cells in Sgca-Null/Scid/Beige Mice.

As previously mentioned, there are no large animal models of LGMD2D and the only available pre-clinical model is the Sgca-null mouse (26). In order to transplant human cells in this dystrophic model, we crossed the immune-deficient scid/beige mouse with the Sgca-null mouse, generating a new dystrophic and immune-deficient triple mutant: the Sgca-null/scid/beige mouse. Phenotypically, Sgca-null/scid/beige mice showed reduced motility and develop kyphosis (FIG. 11C). Histologically, mice showed complete absence of Sgca (FIG. 11D), and typical signs of progressive muscular dystrophy, such as regenerating and necrotic fibers, inflammatory infiltrate and fibrosis (FIG. 11F; confirmed also by elevated creatine kinase levels in FIG. 11G). A detailed analysis is available in the Supplementary Material (See Example 2).

10⁶ MyoD-ER-transduced and genetically corrected LGMD2D HIDEMs were marked with a lentivector expressing GFP and intramuscularly transplanted in the TA muscle of juvenile Sgca-null/scid/beige mice (see Supplementary Material (See Example 2) for details). This resulted in a good colonization, as shown in FIG. 6A, with donor cells inside recipient skeletal muscle fibers as soon as 7 days post-transplantation (FIG. 6B). After 1 month many SGCA+ fibers containing human nuclei were detected (53±14 SEM fibers/tibialis anterior muscle section; FIG. 6C); moreover, reconstitution of the dystrophin-associated protein complex was demonstrated by co-expression of β and γ-sarcoglycans (SGCA and SGCB; FIG. 6C). Intra-arterial transplantation of genetically corrected HIDEMs resulted in colonization of skeletal muscles downstream of the injection site (FIG. 6D), with cells outside the vessels as soon as 12 hours post-injection (FIG. 6D), demonstrating HIDEMs ability to cross the vessel-wall. These data were confirmed by quantitative DNA PCR performed 24 hours after injection, comparing HIDEMs (right leg) with the cells from which they have been derived (left leg) (FIG. 6E) (details in the Supplementary Material (See Example 2)): all HIDEMs showed higher engraftment than pre-reprogramming cells, although we observed variability among different lines. One month after injection SCGA expression was detected by immunofluorescence and RT-PCR analyses (FIG. 6F,G).

Finally, we investigated the possibility of enhancing HIDEM engraftment upon intra-specific transplantation. To this aim, we generated and transplanted murine iPSC-derived MABs (MIDEMs; n=18; FIG. 12), and detected five/six fold more SGCA+ myofibers compared to human cells (SGCA+ myofibers±SEM: 286±41 vs. 53±14; FIG. 6H, I), indicating that there are specie-specific variables other than the adaptive immune system in controlling their engraftment. As a matter of fact, we detected a significant functional amelioration of motor capacity using a treadmill test to exhaustion in animals transplanted with MIDEMs, with mice running approximately one fold more than untreated animals (normalizing performances with their baseline values; FIG. 6J).

### Discussion

Previous studies from our laboratory demonstrated rescue of dystrophic Sgca-null mice by intra-arterial transplantation of murine MABs (22). We then decided to apply this strategy to human, genetically corrected LGMD2D MABs. However, we found that LGMD2D patients have a reduced number of AP+ pericytes in vivo and thus in vitro derivation of MABs was not possible. To overcome this problem, we developed a strategy that allows the derivation and propagation in culture of a population of MABs-like mesodermal stem/progenitor cells (HIDEMs) from iPSCs. The reproducibility of this protocol was validated using 10 different human iPSC lines generated in four different laboratories with different approaches. Notably, potential sources of variation among different HIDEMs (e.g., age, sex and residual pluripotency gene expression) did not correlate with reprogramming and/or differentiation efficiency, confirming recent evidence (27). No significant differences were observed when cMYC was excluded from the reprogramming cocktail and when HIDEMs were derived from viral-integration-free iPSCs, thus adding another layer of safety for their future clinical use. We succeeded in deriving iPSC lines from all the LGMD2D patients subjected to this procedure (4/4), providing also evidence of iPSC generation from human myoblasts (Pt.1), with no apparent differences between healthy and dystrophic fibroblasts. Importantly, LGMD2D HIDEMs were easily transduced with lentiviral vectors, resulting in a genetically corrected, expandable, clonogenic, non-tumorigenic and transplantable cellular population.

In order to test the therapeutic potential of HIDEMs for LGMD2D in vivo, we generated a new dystrophic and immune-deficient model: the Sgca-null/scid/beige mouse. Upon intramuscular and intra-arterial injection, HIDEMs engrafted dystrophic skeletal muscle and gave rise to clusters of SGCA+ myofibers, providing evidence of their similarity with bona fide MABs. Variable levels of engraftment of human cells in mouse dystrophic muscle were observed, possibly related to different levels of inflammation and sclerosis in the host, and to different expression levels of adhesion proteins in different cell population. These differences are currently under investigation. Prospective purification of HIDEMs by pericyte markers, such as AP or CD146, could also be explored in the near future; nevertheless HIDEMs, isolated as described here, never gave rise to tumors upon subcutaneous, intramuscular and intra-arterial transplantation into immune-deficient mice. Taken together, these data demonstrate derivation of an iPSC-derived, expandable and defined stem/progenitor cell population and the feasibility of its genetic correction opens a number of possible scenarios for stem cell-based autologous therapies.

Recent adeno-associated virus-based gene therapy trials showed promising results for LGMD2D (34). Nevertheless, immunity and the loss of transgene expression are still hurdles that need to be overcome by this technology (35, 36). On the other hand, the limited availability of adult, tissue-specific stem/progenitor cells is a relevant obstacle for cell therapies. In such a case, reprogramming followed by lineage-specific commitment/differentiation might solve this problem. This strategy also overcomes one of the main limitations of cell therapy, which is the availability of large amounts of cells for transplantation, raising the possibility of virtually unlimited cell expansion in culture.

Deriving patient-specific iPSCs and expanding their differentiated progeny provide an invaluable tool for gene and cell therapies. Although LGMD2D is a rare genetic disease, it provides a platform to demonstrate the potential of iPSC technology, “reprogramming” lineage-specific commitment from the bench to clinical experimentation for other forms of muscular dystrophy.

### Cell Cultures

Human MABs and HIDEMs were cultured in MegaCell DMEM (Sigma, USA) as described (37). Alternatively, the same cells were cultured in Iscove's Modified Dulbecco's Medium (IMDM; Sigma) containing 10% FBS, 2 mM glutamine, 0.1 mM (3-mercaptoethanol, 1% NEAA, 5 ng/ml human bFGF, 100 IU ml⁻¹ penicillin, 100 mg/ml⁻¹ streptomycin, 0.5 μM oleic and linoleic acids (Sigma), 1.5 μM Iron [II] cloride tetrahydrate (Fe⁺⁺; Sigma), 0.12 μM Iron [III] nitrate nonahydrate (Fe⁺⁺⁺; Sigma) and 1% Insulin/Transferrin/Selenium (Gibco). Instead of 1.5 μM Iron [II] cloride tetrahydrate (Fe⁺⁺; Sigma), 0.12 μM Iron [III] nitrate nonahydrate (Fe⁺⁺⁺; Sigma), Fer-In-Sol (Mead Johnson), 0.12 μM Fe⁺⁺⁺ (Iron [III] nitrate nonahydrate (Sigma) or Ferlixit (Aventis) can also be used.

iPSCs were cultured as described (1, 2, 38). Vector-free episomal human iPSCs (Gibco; A1377) were a certified zero-footprint, viral-integration-free human iPSCS line generated from cord blood-derived CD34+ progenitors using a three plasmid and seven-factor EBNA-based episomal system. The other healthy donor iPSC lines utilized in this study have been described in (38). The murine iPSCs utilized here were described in (33). and were cultured as previously described.

### LGMD2D Samples

LGMD2D skeletal muscle cells and biopsies were obtained from biobanks of Dr.s Maurizio Moggio (Telethon Genetic BioBank Network; Ospedale Maggiore Policlinico, Milan, Italy), Marina Mora (Telethon Genetic BioBank Network; Istituto Neurologico Carlo Besta, Milan, Italy) Benedikt Schoser and Peter Schneiderat (Munich Tissue Culture Collection (MTCC), Friedrich-Baur Institute, Munich, Germany).

We are also grateful to Dr.s Jorge Diaz-Manera (Hospital Santa Creu i Sant Pau, Barcelona, Spain) and Stefano Previtali (San Raffaele Scientific Institute, Milan, Italy) for providing LGMD2D slides.

Reprogramming to iPSCs

Generation of iPSCs from human cells was done using a standard retrovirus-based system previously published (2). Details are available in Supplementary Material (See Example 2).

Generation of iPSC-Derived MAB-Like Cells

Substantial modification of the available protocols to generate vascular cells from ESCs (eg. in(39)) helped in the initial set up of this method. The main steps of the protocol for HIDEM derivation are summarized here:
1. Dissociation of iPSCs colonies to single cell suspension (week 1):
   a. 10 μM Rock inhibitor for 1 hour in iPSC medium (see above).
   b. 30-120 minutes at 37° C., 5% CO₂ in dissociation medium (0.5 mM EDTA, 0.1 mM β-mercaptoethanol, 3% FBS in PBS without Ca²⁺ & Mg²⁺)
   c. Gently shake dishes every 15 minutes to dissociate colonies;
   d. Collect and gently resuspend cells with a P1000 tip to favour dissociation.
2. Seed 6×10⁴/cm² cells obtained in step 1 on a Matrigel™-coated dish (approximately 6×10⁵ cells/3.5 cm dish; week 1) in α-MEM (Gibco) medium containing antibiotics (P/S), 10% FBS, nucleotides and 0.2% β-mercaptoethanol for one week at 37° C., 5% CO₂ and 3-5% O₂.
3. Dissociate culture (as described in step 1), gently scrape dish surface with a cell scraper, filter solution using a 40 μm strainer and seed 2.5×10⁴ cells/cm² with medium and conditions as in step 2 (week 2).
4. If human MAB-like cells are present (see FIG. 3B and (37); wait up to ten days from step 3), trypsinize cells (5 min at 37° C., 5% CO₂ and 3-5% O₂) and seed them on a Matrigel™-coated dish at approximately 80% confluency in human MAB complete media (either MegaCell DMEM or IMDM base, see above; week 3).
5. Split cells (with trypsin from now on) when they reach 100% confluency to have again a culture at 80% confluency, from now on plastic and in human MAB medium (week 3-4).
6. From now on culture HIDEMs exactly like human MABs, as described above and detailed in (37).

Differentiation of murine iPSCs to MIDEMs was done following the above protocol. The main difference with HIDEM generation protocol was the introduction of a purification step after point no. 5 (see above): cells were indeed negatively FACS-sorted for SSEA1, SSEA3 and AP (see below), in particular SSEA-1, to remove residual pluripotent cells

### In Vitro Differentiation Assays

Skeletal and smooth muscle differentiation were performed as previously described (28, 37). Details for embryoid bodies formation and differentiation are available in the Supplementary Material.

### Transplantation

Intramuscular (n=25 Sgca-null/scid/beige mice) and intra-arterial (n=15 Sgca-null/scid/beige mice) injections were done as previously described (19). When MyoD-ER-expressing cells were transplanted, 4OH-tamoxifen was given once a day (intra-peritoneally) for a total of 14 days starting from 1 day prior to transplantation.

### Proliferation Analyses

Growth curves and telomeric repeat amplification protocol (TRAP) has been performed as recently described (19).

### Histology, Histochemistry, Immunofluorescence and Karyotype Analysis

Tissue sections were stained with hematoxylin & eosin (Sigma-Aldrich) according to standard protocols. Masson Trichrome staining was performed following protocol provided from the manufacturer (Bio-Optica, Italy). Alkaline phosphatase was enzymatically detected as already described (14) or by using the standard protocol available with the PermaBlue/AP staining kit (Histo-Line laboratories). Immunofluorescence is detailed in the Supplementary Material (See Example 2). Karyotype analyses were performed and certified by Synlab Diagnostic Services Srl (Italy) using QFQ staining. 50 metaphases/sample have been analyzed.

### PCRs and Immunoblotting

Genotyping PCR for Sgca and scid mutations were done as already described (19, 26) using the following primers. Genotyping PCR for the beige (Lyst^{bg}) mutation was performed by Charles River Laboratories, USA. Quantitative real-time PCRs are detailed in the Supplementary Material (See Example 2). Western blot was performed as already described (19) (details in the Supplementary Material—see Example 2).

### Mice

Scid, Scid/beige, NOD/scid, NSG and nude mice were purchased from Charles River Laboratories and were housed in San Raffaele Scientific Institute animal house together with Sgca-null/scid/beige. All mice were kept in specific pathogen free (SPF) conditions and all procedures involving living animals conformed to Italian law (D.L.vo 116/92 and subsequent additions) and were approved by the San Raffaele Institutional Review Board.

Background strain characterization was performed by Charles River Laboratories, using the Mouse 348 SNP panel.

For generation of Sgca-null/scid/beige mouse, females homozygous for Sgca mutation (Sgca^{-/-}) were bred with homozygous scid/beige^{-/-} males. The resulting F1 heterozygous females were crossed with scid/beige^{-/-} males. In F2 mice (and in subsequent generations), we verified Sgca and scid mutation (beige mutation was genotyped by Charles River laboratories, USA), leucopenia and the absence of B and T lymphocytes. Then we isolated Sgca^{+/-}/scid/beige^{-/-} females and crossed them with scid/beige^{-/-} males for 3 generations. In F5, Sgca^{-/-}scid/beige^{-/-} males and females were bred together to generate mice homozygous for both scid/beige and Sgca mutations. Sgca^{+/+} and Sgca^{-/-} immunocompetent mice, as well as Sgca^{+/+} immune-deficient matched controls were also maintained in the colony. Animals of all genotypes presented an average of 68.7%±2.2 (SD; n=13) of CB 17 background according to Single Nucleotide Polymorphism analysis (Charles River laboratories; data not shown).

### Viral Vectors

Reprogramming retroviruses were produced as already published (2). MyoD-ER construct was kindly provided by Dr. Jeffrey S. Chamberlain (University of Washington School of Medicine, Seattle, USA) and used as previously described (29). Human muscle-specific SGCA lentiviral vector (pLentiMLC1F/SGCA) construction is described in the Supplementary Material (See Example 2).

### Tumorigenic and Teratoma Formation Assays

For HIDEMs tumorigenesis 71 immune-deficient mice (9/HIDEM population [5 scid/beige+4 nude], 4 for HeLa cells as positive control [2 scid/beige+2 nude; see also FIG. 7B] and 4 for human MABs [2 scid/beige+2 nude] as negative control) were injected subcutaneously with 2×10⁶ cells/150 μl of PBS without calcium and magnesium containing 0.2 IU of sodium heparin (Mayne Pharma). No tumors were evident after a minimum of 6 months of follow-up. MIDEMs tumorigenesis was done in 10 scid/beige mice: no tumors were evident after a minimum of 3 months of follow-up.

A detailed description of the teratoma formation assay in available in the Supplementary Material (See Example 2).

### Surface Marker Analysis and Gene Expression Profiling

A detailed description of the procedures, antibodies and meta-analysis is available in the Supplementary Material (See Example 2). Raw data of HIDEM and control human MAB gene expression profiling are going be submitted to GEO repository and will be available for download.

### Motor Capacity and Exercise Tolerance: Time to Exhaustion (Treadmill)

Control untransplanted (vehicle: PBS) Sgca^{+/+}/scid/beige (n=5 for IM; n=8 for IA), untransplanted (vehicle) Sgca-null/scid/beige (n=8 for 1M; n=8 for IA) and transplanted Sgca-null/scid/beige (n=8 for IM; n=5 for IA) were tested for functional recovery on a treadmill (Columbus Instruments, USA), as recently reported (19).

### Statistical Analysis

We expressed values as means±standard error (SEM). We assessed significance of the differences between means by Student's T-test and when more than 2 groups had to be compared we used one-way ANOVA followed by Tukey's post-test to determine which groups were statistically significant. A probability of less than 5% (P<0.05) was considered to be statistically significant. Data were analyzed using Microsoft Excel 12.1 and GraphPad Prism 5.

### Example 2

### Supplementary Characterisation and Generation of Cells

### Cell Cultures

Human MABs and HIDEMs were cultured in MegaCell DMEM (Sigma, USA) containing 5% FBS (Lonza, Switzerland), 2 mM glutamine (Sigma), 0.1 mM (3-mercaptoethanol (Gibco, USA), 1% non essential amino acids (NEAA; Sigma), 5 ng/ml human bFGF (Invitrogen, USA), 100 IU ml⁻¹ penicillin and 100 mg/ml⁻¹ streptomycin (Sigma). Alternatively, the same cells were cultured in Iscove's Modified Dulbecco's Medium (IMDM; Sigma) containing 10% FBS, 2 mM glutamine, 0.1 mM β-mercaptoethanol, 1% NEAA, 5 ng/ml human bFGF, 100 IU ml⁻¹ penicillin, 100 mg/ml⁻¹ streptomycin, 0.5 μM oleic and linoleic acids (Sigma), 1.5 μM Iron [II] cloride tetrahydrate (Fe⁺⁺; Sigma), 0.12 μM Iron [III] nitrate nonahydrate (Fe⁺⁺⁺; Sigma) and 1% Insulin/Transferrin/Selenium (Gibco).

Human iPSCs were cultured on top of a layer of 2×10⁴/cm² Mitomicin-C (Sigma) inactivated mouse embryonic fibroblasts (MEFs) onto growth factor reduced Matrigel™ (BD, USA)-coated dishes. The culture medium used was Knock Out DMEM (KO-DMEM; Gibco) containing 25% Knock Out Serum Replacement (KSR; Gibco), 2 mM L-Glutamine, 1 mM Sodium Pyruvate (Sigma), 100 IU ml⁻¹ penicillin and 100 mg/ml⁻¹ streptomycin, 1% NEAA, 0.2 mM (3-mercaptoethanol and 10 ng/ml human bFGF. Colonies were screened daily for differentiated areas, which were removed when present. When 60-70% of the surface was covered by colonies, they were split (usually 1:5) using collagenase (5 minutes at 37° C.; Gibco), after chopping the surface of the well with a blade to obtain fragmentation of large colonies into small pieces. Colonies were usually pre-treated with 10 μM Rock inhibitor (Y-27632; Calbiochem) in complete iPSC medium for 1 hour before splitting to increase cell survival. Vector-free episomal human iPSCs (Gibco; A1377) were a certified zero-footprint, viral-integration-free human iPSCS line generated from cord blood-derived CD34+ progenitors using a three plasmid and seven-factor (OCT4, SOX2, KLF4, MYC, NANOG, LIN28 and SV40T) EBNA-based episomal system. The other healthy donor iPSC lines utilized in this study have been described in (38). The murine iPSCs utilized here were described in (33), and were cultured as previously described (1).

C2C12 myoblasts were cultured in DMEM (Sigma) containing 20% FBS, 2 mM L-Glutamine, 1 mM Sodium Pyruvate, 100 IU ml⁻¹ penicillin and 100 mg ml⁻¹ streptomycin.

### LGMD2D Samples

LGMD2D skeletal muscle cells and biopsies were obtained from biobanks of Dr.s Miaurizio Moggio (Telethon Genetic BioBank Network; Bank of DNA, Cell Lines and Nerve-Muscle-Cardiac Tissues, Ospedale Maggiore Policlinico Mangiagalli e Regina Elena, Milan, Italy), Marina Mora (Telethon Genetic BioBank Network; Cells, tissues and DNA from patients with neuromuscular diseases, Istituto Neurologico Carlo Besta, Milan, Italy) Benedikt Schoser and Peter Schneiderat (Munich Tissue Culture Collection (MTCC), Friedrich-Baur Institute, Munich, Germany).

We are also grateful to Dr.s Jorge Diaz-Manera (Neurology Department, Hospital Santa Creu i Sant Pau, Universitat Autonoma de Barcelona, Spain) and Stefano Previtali (Institute of Experimental Neurology, Department of Neurology, San Raffaele Scientific Institute, Milan, Italy) for providing LGMD2D slides. See table 1 for additional details.

Reprogramming to iPSCs

Generation of iPSCs from human cells was done using a standard retrovirus-based system previously published (2). Four or three retroviruses (no cMYC for one healthy and one LGMD2D-iPSC line) containing OCT4, KLF4, SOX2 and cMYC were used to infect 2×10⁵ human cells. Two serial retroviral transductions were performed (the second done 24 hours after the first one). 24 hours after the second infection the cells were seeded on top of a layer of 1×10⁶ Mitomicin-C-inactivated MEFs onto a Matrigel™-coated 10 cm dish. After 4 days the culture medium (DMEM with 10% FBS) was replaced daily with complete iPSCs medium (see above). To enhance colony formation, 0.5 mM valproic acid (Sigma) was added for ten days after switching from DMEM to KO-DMEM and cultures were kept at 3% O₂. After 30 days from transduction ESC-like colonies started to appear, which were selected by morphology and expanded in culture. At least 3 clones/patient were grown and characterized.

Generation of iPSC-Derived MAB-Like Cells

Substantial modification of the available protocols to generate vascular cells from ESCs (eg. in (39)) helped in the initial set up of this method. The main steps of the protocol for HIDEM derivation are summarized here:
1. Dissociation of iPSCs colonies to single cell suspension (week 1):
   a. 10 μM Rock inhibitor for 1 hour in iPSC medium (see above).
   b. 30-120 minutes at 37° C., 5% CO₂ in dissociation medium (0.5 mM EDTA, 0.1 mM β-mercaptoethanol, 3% FBS in PBS without Ca²⁺ & Mg²⁺)
   c. Gently shake dishes every 15 minutes to mechanically dissociate colonies;
   d. Collect and gently resuspend cells with a P1000 tip to favour dissociation.
2. Seed 6×10⁴/cm² cells obtained in step 1 on a Matrigel™-coated dish (approximately 6×10⁵ cells/3.5 cm dish; week 1) in α-MEM (Gibco) medium containing antibiotics (P/S), 10% FBS, nucleotides and 0.2% β-mercaptoethanol for one week at 37° C., 5% CO₂ and 3-5% O₂.
3. Dissociate culture (as described in step 1), gently scrape dish surface with a cell scraper, filter solution using a 40 μm strainer and seed 2.5×10⁴ cells/cm² with medium and conditions as in step 2 (week 2).
4. If human MAB-like cells are present (see FIG. 3B and (37); wait up to ten days from step 3), trypsinize cells (5 min at 37° C., 5% CO₂ and 3-5% O₂) and seed them on a Matrigel™-coated dish at approximately 80% confluency in human MAB complete media (either MegaCell DMEM or IMDM base, see above; week 3).
5. Split cells (with trypsin from now on) when they reach 100% confluency to have again a culture at 80% confluency, from now on plastic and in human MAB medium (week 3-4).
6. From now on culture HIDEMs exactly like human MABs, as described above and detailed in (37).

Differentiation of murine iPSCs to MIDEMs was done following the above protocol. The main difference with HIDEM generation protocol was the introduction of a purification step after point no. 5 (see above): cells were indeed negatively FACS-sorted for SSEA1, SSEA3 and AP (see below) to remove residual pluripotent cells

### In Vitro Differentiation Assays

For skeletal muscle differentiation, confluent cultures were exposed to DMEM containing 2% horse serum (EuroClone) for at least 7 days (medium replaced every other day). When MyoD-ER was used, cells were exposed to 1 μM of 4-hydroxy-tamoxifen (4OHT; Sigma) for 24 hours in growth medium and for a further 24 hours in differentiation medium containing DMEM plus 2% horse serum; the cultures were then exposed to differentiation medium for an additional 3 to 5 days. Co-culture assays were done by seeding different ratios of C2C12 myoblasts together with MABs (1:5 to 1:10), as previously described (28, 37). This assay tests the ability of MABs to differentiate into skeletal muscle cells in the presence of an inducer cell line, such as C2C12. 24 hours after mixing and seeding the two cellular populations, the cultures were switched to differentiation medium containing DMEM plus 2% horse serum for 1 week. Terminal differentiation was then analyzed by immunofluorescence staining for myosin heavy chain (MyHC).

Smooth muscle differentiation was induced by TGF-β1 (Sigma), as previously described (28, 37). The final concentration adopted in differentiation medium (DMEM plus 2% horse serum) was 5 ng/ml and differentiation time was 7 days. Morphological change to large, flat and typically elongated cells were evident starting from day 3 to day 4 and differentiation was evident after immunofluorescence staining for α-smooth muscle actin (αSMA).

Vascular-like network formation was done by seeding a two-fold excess of HUVECs with HIDEMs into a Matrigel™ gel sandwich for 4 days in EGM medium (Lonza) containing 20% FBS and VEGF-A, or alternatively HIDEMs alone or in the same medium on Matrigel™-coated coverslips.

For embryoid bodies (EBs) formation and differentiation, iPSCs were harvested with collagenase IV (Invitrogen) for 1 hour and seeded at 1.2×10⁵ cell/cm² in bacterial culture dishes (Sterilin, UK; 3 confluent 3.5 cm dishes of iPSCs colonies per 10 cm bacterial dish) in complete iPSC medium without bFGF supplementation. Five days after, EBs were harvested and seeded onto Matrigel™-coated tissue culture dishes in DMEM with 20% FBS to induce spontaneous differentiation and maintained in culture for 20 days, replacing the medium every other day. Immunofluorescence staining after two weeks of culture revealed nestin (Chemicon, USA; neuroectodermal marker), Sox17 (R&D, USA; endodermal marker) and αSMA (Sigma; mesodermal marker) positive cells outgrowing from EBs, demonstrating their capability of giving rise to cell types of the three embryonic lineages.

### Transplantation

For intra-muscular injections, 2-weeks or 2-months-old Sgca-null/scid/beige mice were used for experiments (n=25). The majority of the mice were trained 24 hours before transplantation. Intra-muscular delivery was done by injecting 10⁶ cells diluted in 30 μl of PBS without calcium and magnesium into TA, Gastrocnemius and Quadriceps (vastus intermedius) muscles using a 30G syringe (BD).

Intra-arterial delivery was done as previously described (19). Briefly, mice (n=15) were anesthetized with an intra-peritoneal injection of Avertin, shaved and disinfected. An incision in the inguinal region was performed, the femoral artery was isolated, 10⁶ cells were diluted in 50 μl of PBS (without calcium and magnesium) containing 10% of 1.25 mg/ml Patent-Blue vital dye (Sigma) and injected into the femoral artery. The wound was then disinfected, closed with sutures and antibiotics (Baytril; Bayer, Germany) and analgesics (Rimadyl, Pfizer, USA) were administered.

When MyoD-ER-expressing cells were transplanted, 4OH-tamoxifen (1 mg/100 μl suspended in 5% ethanol and 95% oil) was given once a day (intra-peritoneally) for a total of 14 days starting from 1 day prior to transplantation.

### Immunofluorescence

Cells were washed with PBS and fixed with 4% paraformaldehyde (Sigma) at room temperature (RT) for 10 minutes, permeabilized with 0.2% Triton X-100 (Sigma) and 1% BSA (Sigma) in PBS for 15 minutes at RT and 10% donkey and/or goat serum (Sigma) was used as blocking solution to reduce secondary antibody background signal. Muscle samples were frozen in liquid nitrogen cooled isopentane (VWR, Italy) and serial 8 μm sections were cut with a cryostat (Leica). Cells and tissue sections were incubated overnight at 4° C. with the following primary antibodies: mouse anti-Sgca (Novocastra, UK; NCL-a-SARC); rabbit anti-Sgca (Sigma; HPA007537); mouse anti-Sgcb (Novocastra, NCL-b-SARC); mouse anti-Sgcg (Novocastra, NCL-g-SARC); rabbit anti-Dystrophin (Sigma, HPA002725); rabbit anti-laminin (Sigma; L9393); mouse anti-myosin heavy chain (MyHC; MF20, Developmental Studies Hybridoma Bank, USA), mouse anti-MyoD 1 (Dako, Denmark; M3512), rabbit anti-EGFP (Molecular Probes; A-11122), chicken anti-GFP (Millipore; AB16901), mouse anti-lamin A/C (Novocastra; NLC-LAM-A/C), rat ant-mouse cd68 (Serotec, UK; MCA1957), rat anti-CD11b (BD Pharmigen; 550282), mouse anti-human cMyc (Roche; 11667149001), rabbit anti-Sox2 (Abcam, USA; ab97959), rabbit anti-Oct4 (Abcam; ab18976), mouse anti-α-smooth muscle actin (Sigma; A2547).

After incubation, samples were washed with 0.2% Triton X100 (Sigma), 1% BSA (Sigma) in PBS (Sigma) and then incubated with the appropriate 488, 546, 594 or 647-fluorochrome conjugated IgGs (Molecular Probes) together with Hoechst 33342 (Fluka, Sigma; B2261) for 1 hour at RT in 0.2% Triton X100-PBS. After three final washes, dishes or slides were mounted using fluorescent mounting medium (Dako) and watched under fluorescent microscopes (Nikon, Japan and Leica). Images were analyzed using ImageJ (NIH) and PhotoshopCS (Adobe) softwares.

### Proliferation Analyses

Growth curves and telomeric repeat amplification protocol (TRAP) has been performed as recently described (19).

### Histology, Histochemistry and Karyotype Analysis

Tissue sections were stained with hematoxylin & eosin (Sigma-Aldrich) according to standard protocols. Masson Trichrome staining was performed following protocol provided from the manufacturer (Bio-Optica, Italy). Alkaline phosphatase was enzymatically detected as already described (14) or by using the standard protocol available with the PermaBlue/AP staining kit (Histo-Line laboratories).

Karyotype analyses were performed and certified by Synlab Diagnostic Services Srl (Italy) using QFQ staining. 50 metaphases/sample have been analyzed.

### PCRs

Genotyping PCR for Sgca and scid mutations were done as already described (19, 26) using the following primers:

Sgca:
INT1 in intron 1:
(SEQ ID NO: 1)
5′-CAGGGCTGGGAGCTGGGTTCTG-3′;
EX2 in intron 3:
(SEQ ID NO: 2)
5′-CCCAGGGCCTTGATGCCT-3′;
(deleted in the null allele)
NEOTR:
(SEQ ID NO: 3)
5′-GCTATCAGGACATAGCGTTGGCTA-3′;
Scid (Prkdc^{scid}):
F (SEQ ID NO: 4):
(forward)
(SEQ ID NO: 4)
5′GAGAAAAGGAGGATCATGGATTCAAGAAATAAATGTAACG-3′;
WR:
(reverse)
(SEQ ID NO: 5)
5′-TGGCCCCTGCTAACTTTCTCTCTTAGCA-3′;
MF:
(forward)
(SEQ ID NO: 6)
5′-TGGTATCCACAACATAAAATACGCTAA-3′;
R:
(reverse)
(SEQ ID NO: 7)
5′-CCTAAGAGTCACTTTCTCCATTTACACAGTGAAGTGCC-3′;
Genotyping PCR for the beige (Lyst^{bg}) mutation
was performed by Charles River Laboratories, USA.

For RT-PCR, one microgram of RNA, extracted with the RNeasy mini kit (QIAGEN) from cells or with TRIZOL (Invitrogen) from tissues, was converted into double-stranded cDNA with the cDNA synthesis kit ImProm™-II Reverse Trascription System (Promega), according to the manufacturer's instructions. Dystrophin primers are available in (19) and the other primers used are:

SGCA:
(forward)
(SEQ ID NO: 8)
5′-GCCTCCACTTCTGTCTTGCT-3′;
(reverse)
(SEQ ID NO: 9)
5′-CCACCAAGAAGTCACGGTCT-3′.
MYOD:
(forward)
(SEQ ID NO: 10)
5′-CACTCAAGCGCTGCACGTCG-3′;
(reverse)
(SEQ ID NO: 11)
5′-GGCCGCTGTAGTCCATCATGC-3′.
MYOGENIN:
(forward)
(SEQ ID NO: 12)
5′-CCAGGGGTGCCCAGCGAATG-3′;
(reverse)
(SEQ ID NO: 13)
5′-AGCCGTGAGCAGATGATCCCC.
GAPDH:
(forward)
(SEQ ID NO: 14)
5′-TTCACCACCATGGAGAAGGC-3′;
(reverse)
(SEQ ID NO: 15)
5′-GGCATGGACTGTGGTCATGA-3′.

Quantitative real-time PCRs for reprogramming factors were performed with SsoFast™ EvaGreen® Supermix (BioRad), according manufacturer's instruction using following primers:

OCT4:
(forward)
(SEQ ID NO: 16)
5′-ATGCACAACGAGAGGATTTTGA-3′;
(reverse)
(SEQ ID NO: 17)
5′-CTTTGTGTTCCCAATTCCTTCC-3′;
SOX2:
(forward)
(SEQ ID NO: 18)
5′-TTACCTCTTCCTCCCACTCCAG-3′;
(reverse)
(SEQ ID NO: 19)
5′-GGGTTTTCTCCATGCTGTTTCT-3′;
KLF4:
(forward)
(SEQ ID NO: 20)
5′-ACCCACACAGGTGAGAAACCTT-3′;
(reverse)
(SEQ ID NO: 21)
5′-GTTGGGAACTTGACCATGATTG-3′.
β-ACTIN:
(forward)
(SEQ ID NO: 22)
5′-ACCATTGGCAATGAGCGGTTC-3′;
(reverse)
(SEQ ID NO: 23)
5′-CACTTCATGATGGAGTTGAAGG-3′.
pMXs.REV:
(SEQ ID NO: 24)
5′-CCCTTTTTCTGGAGACTAAATAAA-3′
(used as a reverse primer with the OCT4,
SOX2 and KLF4 forward primers to detect
expression exogenous/viral transgenes);

Other real-time quantitative PCR were performed with a real-time PCR thermocycler (Mx3000P; Stratagene). RNA was retro-transcribed as described above and each cDNA sample was amplified in triplicate using GoTaq® qPCR master mix (Promega). The following primers were utilized:

GFP:
(forward)
(SEQ ID NO: 25)
5′-CGGTCACGAACTCCAGCA-3′;
(reverse)
(SEQ ID NO: 26)
5′-ACAAGCAGAAGAACGGCATC-3′;;
GAPDH:
(forward)
(SEQ ID NO: 27)
5′-CCATCTTCCAGGAGCGAGA-3′;
(reverse)
(SEQ ID NO: 28)
5′-TGTCATACCAGGAAATGAGC-3′.
TELOMERASE:
(forward)
(SEQ ID NO: 29)
5′-GGCACACGTGGCTTTTCG-3′;
(reverse)
(SEQ ID NO: 30)
5′-GGTGAACCTCGTAAGTTTATGCAA-3′.
CHD7:
(forward)
(SEQ ID NO: 31)
5′-CAGGGCAGTATTCTCGATATC-3′.;
(reverse)
(SEQ ID NO: 32)
5′-GCATTGGGGTATCTTGGTAC-3′..

### Immunoblotting

Western blot was performed as already described (19). Briefly, proteins were extracted from both cells and tissues using RIPA buffer plus protease inhibitors. Protein concentrations were determined by BCA protein assay (Pierce) using bovine serum albumin as standard. 50 μg of proteins were loaded for SGCA detection. Total homogenates were separated by SDS polyacrylamide gel electrophoresis (SDS-PAGE) and then transferred to Amersham membranes, saturated with 5% milk in 0.1% Tween-20 (Sigma)-PBS and hybridized overnight at 4° C. with the following antibodies: mouse anti-Sgca (Novocastra; NCL-a-SARC) and mouse anti-GAPDH (Covance; MMS-411R). The filters were then washed and reacted with the proper HRP-conjugated IgGs (Amersham, USA; 1:1000 dilution) for 1 hour at RT and finally visualized with the ECL immunoblotting detection system (Amersham).

### Mice

Scid, Scid/beige, NOD/scid, NSG and nude mice were purchased from Charles River Laboratories and were housed in San Raffaele Scientific Institute animal house together with Sgca-null/scid/beige. All mice were kept in specific pathogen free (SPF) conditions and all procedures involving living animals conformed to Italian law (D.L.vo 116/92 and subsequent additions) and were approved by the San Raffaele Institutional Review Board.

Animals were genotyped as described above. Background strain characterization was performed by Charles River Laboratories, using the Mouse 348 SNP panel. Immunodeficiency was confirmed by determining leukocyte counts with a haemocytometer (Sysmex, model k×21n): scid mice had usually less than 4−5×10³ white blood cells/μl.

### Generation and Characterization of Sgca-Null/Scid/Beige Mouse.

Females homozygous for Sgca mutation (Sgca^{-/-}) were bred with homozygous scid/beige^{-/-} males. The resulting F1 heterozygous females were crossed with scid/beige^{-/-} males. In F2 mice (and in subsequent generations), we verified Sgca and scid mutation (beige mutation was genotyped by Charles River laboratories, USA), leucopenia and the absence of B and T lymphocytes. Then we isolated Sgca^{+/-}/scid/beige^{-/-} females and crossed them with scid/beige^{-/-} males for 3 generations. In F5, Sgca^{-/-}scid/beige^{-/-} males and females were bred together to generate mice homozygous for both scid/beige and Sgca mutations. Sgca^{+/+} and Sgca^{-/-} immunocompetent mice, as well as Sgca^{+/+} immune-deficient matched controls were also maintained in the colony. Animals of all genotypes presented an average of 68.7%±2.2 (SD; n=13) of CB17 background according to Single Nucleotide Polymorphism analysis (Charles River laboratories; data not shown).

Phenotypically, Sgca-null/scid/beige mice show reduced motility and develop kyphosis (FIG. 11C). Histologically, mice show complete absence of Sgca (FIG. 11D; confirmed by western blot in FIG. 11E), typical signs of progressive muscular dystrophy, such as regenerating and necrotic fibers, inflammatory infiltrate and fibrosis (FIG. 11F; confirmed also by elevated creatine kinase levels in FIG. 11G). These features are comparable with the immunocompetent Sgca-null model; however, Sgca-null/scid/beige mice appear to have a slightly more severe dystrophic histopathology than the immunocompetent counterpart, in particular for the fibrosis. The latter finding is also in line with their reduced life-span (FIG. 11H). Sgca-null mice do not have revertant fibers like the mdx model. Indeed we tested Engraftment of adult human MABs was demonstrated by intramuscular and intra-arterial (femoral artery) injection (FIG. 11I). Transplants were infiltrated by host cells, mainly CD68 positive macrophages after 7 days, resulting in a reduction of donor cell engraftment (FIG. 11I). This problem was addressed transplanting younger mice (2 weeks-old), mainly because of a reduced inflammation related to the early stage of muscular dystrophy, and a more “immature” innate immunity, which enhances engraftment of xenotransplants in rodents (43). This strategy resulted in a 8-10 fold increase of grafted human MABs, with reduced macrophage infiltration (FIG. 111).

### Viral Vectors

Reprogramming retroviruses were produced as already published (2). MyoD-ER construct was kindly provided by Dr. Jeffrey S. Chamberlain (University of Washington School of Medicine, Seattle, USA) and used as previously described (29). Working concentrations were determined by quantitative real-time PCR viral titration and myogenic conversion of 10t1/2 cells.

Human muscle-specific SGCA lentiviral vector (pLentiMLC1F/SGCA) was constructed as follow. Coding sequence of human α-sarcoglycan (SGCA) was amplified using pCMV-SPORT6/α-SG as a template (Open Biosystem). SGCA cDNA was amplified with primers having 5′-BamHI flanking sequences. pLenti/MLC1F was digested with BamHI and used for ligation mix with SGCA cDNA. Positive clones were screened with PureLink MiniPrep Kit (Invitrogen) and successfully sequenced.

Lentiviral particles were produced by transient transfection of the vector of interest in association with the packaging vectors (pREV, pD8.74 and pVSV-G) in HEK293T. After 48 hours, culture medium from transfected cells was filtered with a 0.45 mm filter and 100-times concentrated after centrifugation at 20,000 rpm for 2 hrs (at 20° C.).

### Tumorigenic and Teratoma Formation Assays

For HIDEMs tumorigenesis 71 immune-deficient mice (9/HIDEM population [5 scid/beige+4 nude], 4 for HeLa cells as positive control [2 scid/beige+2 nude; see also FIG. 7B] and 4 for human MABs [2 scid/beige+2 nude] as negative control) were injected subcutaneously in the dorsal flank with 2×10⁶ cells/150 μl of PBS without calcium and magnesium containing 0.2 IU of sodium heparin (Mayne Pharma). No tumors were evident after a minimum of 6 months of follow-up.

MIDEMs tumorigenesis was done in 10 scid/beige mice: no tumors were evident after a minimum of 3 months of follow-up.

For teratoma formation, sub-confluent colonies were pre-treated with 10 μM Rock inhibitor for 1 hour in 3.5 cm dishes, chopped and harvested by collagenase treatment followed by surface scraping, resuspended in PBS without calcium and magnesium (50 μl/dish) and finally mixed with an equal volume of a chilled (4° C.) solution of Matrigel™ diluted 1:10 in KO-DMEM. One confluent 3.5 cm dish/mouse (NOD/scid) was administered sub-cutaneously (100 μl final volume) using a pre-chilled (to avoid Matrigel™ polymerization) syringe with a 21G needle. Mice were screened weekly for the presence of growing sub-cutaneous masses, which became evident from 12 weeks after the injection.

### Flow Cytometry

Cells were harvested and resuspended in 1 ml of a solution containing 1% FBS and 2 mM EDTA in PBS and incubated with specific antibodies for 1 hour at 4° C. After washing with PBS, cells were fixed in 2% PFA. Analysis was performed on at least 10,000 events for each sample and determined using a FACScalibur flow cytometer (BD). The acquisition was performed using CELLQUEST software (BD) and analyzed using FCS-express software. A primary gate based on physical parameters (forward and side light scatter, FSC and SSC, respectively) was set to exclude dead cells or small debris. The background level was estimated analyzing the signal of the fluorochrome-conjugated appropriate IgG isotype control. Antibodies used were: anti-CD13 (ID Labs inc.; IDAC1071), anti-CD31 (ID Labs inc.; IDAC1400), anti-CD44 (BD; 553133), anti-CD45 (BD; 555483), anti-CD49b (BD; 553858), anti-CD146 (Biocytex; 5050-PE100T), anti-L-alkaline phosphatase (Santa Cruz; sc-21708), anti-CD56 (Biolegend; 304604), anti-Flk-1 (BD; 555308), anti-SSEA4 (BD; 560128), anti-SSEA1 (BD; 560142), anti-Sca1 (BD; 553336), anti-CD34 (BD; 551387); rat anti-SSEA3 (Santacruz; sc-73066); goat anti mouse AP (R&D; AF2910). Data in FIG. 2A were validated by double staining and FACS analysis for the four possible phenotypes.

### Creatine Kinase (CK) Measurement

Approximately 0.15 ml of blood was collected by tail-vein bleeding using 0.5 ml tubes containing 0.01 ml of 0.12 M EDTA. Serum was prepared by centrifugation and particular care was applied to avoid haemolysis, as it interferes with the assay. CK was then measured by an UV method following standard protocol instructions (CK NAC-activated (CK-NAC), Randox). 5 mice per group were analyzed.

### Gene Expression Profiling, Data Analysis and Meta-Analysis

Total cellular RNA was isolated from HIDEMs and MABs using RNeasy RNA isolation kit (Qiagen, USA) following manufacturer's recommendations. Disposable RNA chips (Agilent RNA 6000 Nano LabChip kit) were used to determine the concentration and purity/integrity of RNA samples using Agilent 2100 bioanalyzer. cDNA synthesis, biotin-labeled target synthesis, HG-U133 plus 2.0 GeneChip (Affymetrix, USA) arrays hybridization, staining and scanning were performed according to the standard protocol supplied by Affymetrix. Datasets for meta-analysis were downloaded from GEO public repository (http://www.ncbi.nlm.nih.gov/geo/). GEO series and samples, along with sample info are available upon request.

Probe level data were normalized and converted to expression values using robust multi-array average (RMA) procedure or DChip procedure (invariant set). Quality control assessment was performed using different Bioconductor packages such as R-AffyQC Report, R-Affy-PLM, R-RNA Degradation Plot. Low quality samples were removed from analysis. Sample data were then filtered in order to remove probe-sets having a standard deviation/mean ratio greater the 0.8 and less that 1000. Principal Component Analysis (PCA) as well as the unsupervised hierarchical clustering were performed using Partek GS®. The agglomerative hierarchical clustering was performed using the Euclidean distance and the average linkage method. Raw data of HIDEM and control human MAB gene expression profiling are going be submitted to GEO repository and will be available for download.

### Motor Capacity and Exercise Tolerance: Time to Exhaustion (Treadmill)

Control untransplanted (vehicle: PBS) Sgca^{+/+}/scid/beige (n=5 for IM; n=8 for IA), untransplanted (vehicle) Sgca-null/scid/beige (n=8 for 1M; n=8 for IA) and transplanted Sgca-null/scid/beige (n=8 for IM; n=5 for IA) were tested for functional recovery on a treadmill (Columbus Instruments, USA), as recently reported (19). Briefly, mice were trained to the procedure (10 minutes every other day; 6 meters/minute) for 1 week. Transplantations were done with 10⁶ HIDEMs/muscle or femoral artery 24 hours after exercise. For the test mice were kept into a 10° inclined treadmill at 6 meters/minute, then the speed was increased 2 meters/minute every 2 minutes until exhaustion, defined as 10 seconds on the shocker plate without attempting to reengage the treadmill. Data are shown as absolute numbers and are also normalized to the running time of the different mice before transplantation (percentages relative to baseline performances).

### Statistical Analysis

We expressed values as means±standard error (SEM). We assessed significance of the differences between means by Student's T-test and when more than 2 groups had to be compared we used one-way ANOVA followed by Tukey's post-test to determine which groups were statistically significant. A probability of less than 5% (P<0.05) was considered to be statistically significant. Data were analyzed using Microsoft Excel 12.1 and GraphPad Prism 5.

TABLE 1
Characteristics of LGMD2D patients and their relative samples.

| Patient | | Muscle | Age at | | | Muscle of |
|---|---|---|---|---|---|---|
| number | Cells | sections | biopsy | Sex | SGCA mutation(s) | origin |
| 1 | Yes | Yes | 32 | Male | Homozygote, cDNA 935, | Quadriceps |
| | | | | | T > G, Met312Arg | |
| 2 | Yes | Yes | 14 | Female | Not available | Quadriceps |
| 3 | Yes | Yes | 10 | Female | Compound heterozygote, | Quadriceps |
| | | | | | cDNA 92 C > T/cDNA 850 | |
| | | | | | C > T, Leu31Pro/Arg284Cys | |
| 4 | Yes | Yes | 10 | Male | Homozygote, exon 3, 229 | Tibialis |
| | | | | | C > T, Arg77Cys | anterior |
| 5 | Yes | Yes | 54 | Male | Compound heterozygote, | Biceps |
| | | | | | nonsense Tyr134 - Stop ex5 | brachii |
| | | | | | and Thr208Ala ex6 | |
| 6 | Yes | Yes | 44 | Female | Compound heterozygote, | Tibialis |
| | | | | | Arg77Cys; Arg284Cys | anterior |
| 7 | No | Yes | NA | Female | Not available | Not |
| | | | | | | available |
| 8 | No | Yes | 45 | Female | Not available | Quadriceps |

### Example 3

### Pluripotent Stem Cell-Derived Mesoangioblasts and Methodological Refinements

Our method to derive MABs from human iPS cells has also been extended to the derivation of MABs from human ES cells and mouse ES cells. In this context, the already described protocol for generating mouse iPS cell-derived MABs has been slightly modified and refined. Cells (both ES and iPS) have been negatively FACS-sorted twice for SSEA1 after the final step of the protocol. This double purification procedure allows increasing the efficiency of the elimination of potentially undifferentiated and tumorigenic cells. Moreover, these cells can be further purified and enriched in their MAB-like fraction by means of positive FACS-sorting for CD34, Sca1 and CD44 (in general, we consider the presence of two out of three of these markers as a required outcome if this additional step is being performed). Three novel cell lines have been generated (one iPS cell-derived and two ES cell-derived) and transplanted subcutaneously into immunodeficient mice to evaluate their safety (absence of tumorigenic potential; n=15 scid/beige mice; 5/line). No mass was identifiable after one month or two months from the injection.

### Generation of Human Embryonic Stem (ES) Cell-Derived MABs (HEDEMs)

In order to extend the method developed with human iPS cells also to human pluripotent stem cells, we have subjected human embryonic stem (ES) cells to the same protocol. Two independent human ES cell lines (Shef-3 and Shef-6) have been amplified (FIG. 13A), seeded and sequentially passaged following the procedure recently described for iPS cells (42). The derived progeny (HEDEMs: human ES cell-derived MAB-like cells) had morphology comparable to that of adult MABs and HIDEMs (FIG. 13B) (42). FACS analysis showed a surface marker profile similar to that of adult MABs and HIDEMs, with the only difference of variable amounts of CD56 (detectable in HEDEMs and not detectable in HIDEMs) and CD13 (reduced in HEDEMs vs. HIDEMs; FIG. 13C). Notably, similarly to HIDEMs, HEDEMs underwent robust myogenic differentiation following infection with the MyoD-ER lentivector and administration of 4OH-tamoxifen. Indeed, FIG. 13D shows large hypertrophic multinucleated myotubes derived from HEDEMs after 7 days of differentiation.

### Example 4

### Reduction of Fibrosis, Increased Force of Contraction and Contribution to the Progenitor Pool Upon Transplantation of MIDEMs

A reduction in fibrotic-adipose tissue in muscles transplanted with MIDEMs was observed (26.24% less than nontransplanted muscle, P<0.05, n=6; FIG. 14A). We also measured the tetanic force of the tibialis anterior muscle and force of contraction on isolated muscle fibers 4 months after transplantation. FIG. 14B shows that in tibialis anterior muscles from mice transplanted intramuscularly and intra-arterially, the tetanic force was significantly higher than in un-treated mice (67% and 83%, respectively; P<0.05). Individual muscle fibers (n=119) were then dissected from the gastrocnemius muscle of the same mice, and the analysis demonstrated that GFP+ myofibers devel-oped greater force than did muscle fibers from untreated mice (FIG. 14B). Finally, to determine whether the transplanted cells were able to contribute to the pool of AP+ pericytes in vivo, we searched for AP+ and GFP+ MIDEMs in the skeletal muscle interstitium of Sgca-null/scid/beige mice. As shown in FIG. 14C, double-positive donor cells were clearly identifiable near GFP+ myofibers, indicating donor cell contribution to muscle regeneration together with replenishment of the pericyte niche in vivo. Notably, the number of AP+ cells per tibialis anterior section was higher than that observed in untreated Sgca-null/scid/beige mice (535.5±39.56 versus 344±36.8, mean±SEM; n=6; P<0.05) and was closer to the number of AP+ cells in wild-type animals (666.5±47.6; n=3; FIG. 14C).

### REFERENCES

1. K. Takahashi, S. Yamanaka, Induction of pluripotent stem cells from mouse embryonic and adult fibroblast cultures by defined factors. Cell 126, 663-676 (2006).
2. K. Takahashi, K. Tanabe, M. Ohnuki, M. Narita, T. Ichisaka, K. Tomoda, S. Yamanaka, Induction of pluripotent stem cells from adult human fibroblasts by defined factors. Cell 131, 861-872 (2007).
3. E. Kiskinis, K. Eggan, Progress toward the clinical application of patient-specific pluripotent stem cells. J Clin Invest 120, 51-59 (2010).
   4. S. M. Wu, K. Hochedlinger, Harnessing the potential of induced pluripotent stem cells for regenerative medicine. Nat Cell Biol 13, 734 (2011).
   5. H. Chang, M. Yoshimoto, K. Umeda, T. Iwasa, Y. Mizuno, S. Fukada, H. Yamamoto, N. Motohashi, Y. Miyagoe-Suzuki, S. Takeda, T. Heike, T. Nakahata, Generation of transplantable, functional satellite-like cells from mouse embryonic stem cells. Faseb J 23, 1907-1919 (2009).
   6. Y. Mizuno, H. Chang, K. Umeda, A. Niwa, T. Iwasa, T. Awaya, S. I. Fukada, H. Yamamoto, S. Yamanaka, T. Nakahata, T. Heike, Generation of skeletal muscle stem/progenitor cells from murine induced pluripotent stem cells. FASEB J, (2010).
   7. T. Barberi, M. Bradbury, Z. Dincer, G. Panagiotakos, N. D. Socci, L. Studer, Derivation of engraftable skeletal myoblasts from human embryonic stem cells. Nat Med 13, 642-648 (2007).
   8. F. S. Tedesco, A. Dellavalle, J. Diaz-Manera, G. Messina, G. Cossu, Repairing skeletal muscle: regenerative potential of skeletal muscle stem cells. J Clin Invest 120, 11-19 (2010).
   9. H. Sakurai, Y. Okawa, Y. Inami, N. Nishio, K. Isobe, Paraxial mesodermal progenitors derived from mouse embryonic stem cells contribute to muscle regeneration via differentiation into muscle satellite cells. Stem Cells 26, 1865-1873 (2008).
   10. R. Darabi, K. Gehlbach, R. M. Bachoo, S. Kamath, M. Osawa, K. E. Kamm, M. Kyba, R. C. Perlingeiro, Functional skeletal muscle regeneration from differentiating embryonic stem cells. Nat Med 14, 134-143 (2008).
   11. R. Darabi, W. Pan, D. Bosnakovski, J. Baik, M. Kyba, R. C. Perlingeiro, Functional Myogenic Engraftment from Mouse iPS Cells. Stem Cell Rev, (2011).
   12. R. Darabi, F. N. Santos, A. Filareto, W. Pan, R. Koene, M. A. Rudnicki, M. Kyba, R. C. Perlingeiro, Assessment of the myogenic stem cell compartment following transplantation of Pax3/Pax7-induced embryonic stem cell-derived progenitors. Stem Cells 29, 777-790 (2011b).
   13. M. Quattrocelli, G. Palazzolo, G. Floris, P. Schoffski, L. Anastasia, A. Orlacchio, T. Vandendriessche, M. K. Chuah, G. Cossu, C. Verfullie, M. Sampaolesi, Intrinsic cell memory reinforces myogenic commitment of pericyte-derived iPSCs. J Pathol 223, 593-603 (2011).
   14. A. Dellavalle, M. Sampaolesi, R. Tonlorenzi, E. Tagliafico, B. Sacchetti, L. Perani, A. Innocenzi, B. G. Galvez, G. Messina, R. Morosetti, S. Li, M. Belicchi, G. Peretti, J. S. Chamberlain, W. E. Wright, Y. Torrente, S. Ferrari, P. Bianco, G. Cossu, Pericytes of human skeletal muscle are myogenic precursors distinct from satellite cells. Nat Cell Biol 9, 255-267 (2007).
   15. A. Dellavalle, G. Maroli, D. Covarello, E. Azzoni, A. Innocenzi, L. Perani, S. Antonini, R. Sambasivan, S. Brunelli, S. Tajbakhsh, G. Cossu, Pericytes resident in postnatal skeletal muscle differentiate into muscle fibres and generate satellite cells. Nat Commun 2, 499 (2011).
   16. M. Sampaolesi, S. Blot, G. D'Antona, N. Granger, R. Tonlorenzi, A. Innocenzi, P. Mognol, J. L. Thibaud, B. G. Galvez, I. Barthelemy, L. Perani, S. Mantero, M. Guttinger, O. Pansarasa, C. Rinaldi, M. G. Cusella De Angelis, Y. Torrente, C. Bordignon, R. Bottinelli, G. Cossu, Mesoangioblast stem cells ameliorate muscle function in dystrophic dogs. Nature 444, 574-579 (2006).
   17. J. Diaz-Manera, T. Touvier, A. Dellavalle, R. Tonlorenzi, F. S. Tedesco, G. Messina, M. Meregalli, C. Navarro, L. Perani, C. Bonfanti, I. Illa, Y. Torrente, G. Cossu, Partial dysferlin reconstitution by adult murine mesoangioblasts is sufficient for full functional recovery in a murine model of dysferlinopathy. Cell Death Dis 1, e61 (2010).
   18. C. Gargioli, M. Coletta, F. De Grandis, S. M. Cannata, G. Cossu, PlGF-MMP-9-expressing cells restore microcirculation and efficacy of cell therapy in aged dystrophic muscle. Nat Med 14, 973-978 (2008).
   19. F. S. Tedesco, H. Hoshiya, G. D'Antona, M. F. Gerli, G. Messina, S. Antonini, R. Tonlorenzi, S. Benedetti, L. Berghella, Y. Torrente, Y. Kazuki, R. Bottinelli, M. Oshimura, G. Cossu, Stem cell-mediated transfer of a human artificial chromosome ameliorates muscular dystrophy. Sci Transl Med 3, 96ra78 (2011).
   20. S. E. Berry, J. Liu, E. J. Chaney, S. J. Kaufman, Multipotential mesoangioblast stem cell therapy in the mdx/utrn−/− mouse model for Duchenne muscular dystrophy. Regen Med 2, 275-288 (2007).
   21. B. G. Galvez, M. Sampaolesi, S. Brunelli, D. Covarello, M. Gavina, B. Rossi, G. Constantin, Y. Torrente, G. Cossu, Complete repair of dystrophic skeletal muscle by mesoangioblasts with enhanced migration ability. J Cell Biol 174, 231-243 (2006).
   22. M. Sampaolesi, Y. Torrente, A. Innocenzi, R. Tonlorenzi, G. D'Antona, M. A. Pellegrino, R. Barresi, N. Bresolin, M. G. De Angelis, K. P. Campbell, R. Bottinelli, G. Cossu, Cell therapy of alpha-sarcoglycan null dystrophic mice through intra-arterial delivery of mesoangioblasts. Science 301, 487-492 (2003).
   23. A. E. Emery, The muscular dystrophies. Lancet 359, 687-695 (2002).
   24. K. Bushby, Diagnosis and management of the limb girdle muscular dystrophies. Pract Neurol 9, 314-323 (2009).
   25. M. Cassano, A. Dellavalle, F. S. Tedesco, M. Quattrocelli, S. Crippa, F. Ronzoni, A. Salvade, E. Berardi, Y. Torrente, G. Cossu, M. Sampaolesi, Alpha sarcoglycan is required for FGF-dependent myogenic progenitor cell proliferation in vitro and in vivo. Development, (2011).
   26. F. Duclos, V. Straub, S. A. Moore, D. P. Venzke, R. F. Hrstka, R. H. Crosbie, M. Durbeej, C. S. Lebakken, A. J. Ettinger, J. van der Meulen, K. H. Holt, L. E. Lim, J. R. Sanes, B. L. Davidson, J. A. Faulkner, R. Williamson, K. P. Campbell, Progressive muscular dystrophy in alpha-sarcoglycan-deficient mice. J Cell Biol 142, 1461-1471 (1998).
   27. G. L. Boulting, E. Kiskinis, G. F. Croft, M. W. Amoroso, D. H. Oakley, B. J. Wainger, D. J. Williams, D. J. Kahler, M. Yamaki, L. Davidow, C. T. Rodolfa, J. T. Dimos, S. Mikkilineni, A. B. MacDermott, C. J. Woolf, C. E. Henderson, H. Wichterle, K. Eggan, A functionally characterized test set of human induced pluripotent stem cells. Nat Biotechnol 29, 279-286 (2011).
   28. M. G. Minasi, M. Riminucci, L. De Angelis, U. Borello, B. Berarducci, A. Innocenzi, A. Caprioli, D. Sirabella, M. Baiocchi, R. De Maria, R. Boratto, T. Jaffredo, V. Broccoli, P. Bianco, G. Cossu, The meso-angioblast: a multipotent, self-renewing cell that originates from the dorsal aorta and differentiates into most mesodermal tissues. Development 129, 2773-2783 (2002).
   29. E. Kimura, J. J. Han, S. Li, B. Fall, J. Ra, M. Haraguchi, S. J. Tapscott, J. S. Chamberlain, Cell-lineage regulated myogenesis for dystrophin replacement: a novel therapeutic approach for treatment of muscular dystrophy. Hum Mol Genet. 17, 2507-2517 (2008).
   30. D. Huangfu, R. Maehr, W. Guo, A. Eijkelenboom, M. Snitow, A. E. Chen, D. A. Melton, Induction of pluripotent stem cells by defined factors is greatly improved by small-molecule compounds. Nat Biotechnol 26, 795-797 (2008).
   31. Y. Yoshida, K. Takahashi, K. Okita, T. Ichisaka, S. Yamanaka, Hypoxia enhances the generation of induced pluripotent stem cells. Cell Stem Cell 5, 237-241 (2009).
   32. H. N. Nguyen, B. Byers, B. Cord, A. Shcheglovitov, J. Byrne, P. Gujar, K. Kee, B. Schule, R. E. Dolmetsch, W. Langston, T. D. Palmer, R. R. Pera, LRRK2 mutant iPSC-derived DA neurons demonstrate increased susceptibility to oxidative stress. Cell Stem Cell 8, 267-280 (2011).
   33. Y. Kazuki, M. Hiratsuka, M. Takiguchi, M. Osaki, N. Kajitani, H. Hoshiya, K. Hiramatsu, T. Yoshino, K. Kazuki, C. Ishihara, S. Takehara, K. Higaki, M. Nakagawa, K. Takahashi, S. Yamanaka, M. Oshimura, Complete genetic correction of ips cells from Duchenne muscular dystrophy. Mol Ther 18, 386-393 (2010).
   34. J. R. Mendell, L. R. Rodino-Klapac, X. Q. Rosales, B. D. Coley, G. Galloway, S. Lewis, V. Malik, C. Shilling, B. J. Byrne, T. Conlon, K. J. Campbell, W. G. Bremer, L. E. Taylor, K. M. Flanigan, J. M. Gastier-Foster, C. Astbury, J. Kota, Z. Sahenk, C. M. Walker, K. R. Clark, Sustained alpha-sarcoglycan gene expression after gene transfer in limb-girdle muscular dystrophy, type 2D. Ann Neurol 68, 629-638 (2010).
   35. J. R. Mendell, K. Campbell, L. Rodino-Klapac, Z. Sahenk, C. Shilling, S. Lewis, D. Bowles, S. Gray, C. Li, G. Galloway, V. Malik, B. Coley, K. R. Clark, J. Li, X. Xiao, J. Samulski, S. W. McPhee, R. J. Samulski, C. M. Walker, Dystrophin immunity in Duchenne's muscular dystrophy. N Engl J Med 363, 1429-1437 (2010).
   36. F. Mingozzi, J. J. Meulenberg, D. J. Hui, E. Basner-Tschakarj an, N. C. Hasbrouck, S. A. Edmonson, N. A. Hutnick, M. R. Betts, J. J. Kastelein, E. S. Stroes, K. A. High, AAV-1-mediated gene transfer to skeletal muscle in humans results in dose-dependent activation of capsid-specific T cells. Blood 114, 2077-2086 (2009).
   37. R. Tonlorenzi, A. Dellavalle, E. Schnapp, G. Cossu, M. Sampaolesi, Isolation and characterization of mesoangioblasts from mouse, dog, and human tissues. Curr Protoc Stem Cell Biol Chapter 2, Unit 2B 1 (2007).
   38. B. Di Stefano, S. M. Maffioletti, B. Gentner, F. Ungaro, G. Schira, L. Naldini, V. Broccoli, A microRNA-Based System for Selecting and Maintaining the Pluripotent State in Human Induced Pluripotent Stem Cells. Stem Cells 29, 1684-1695 (2011).
   39. I. Goldberg-Cohen, G. Beck, A. Ziskind, J. Itskovitz-Eldor, Vascular cells. Methods Enzymol 418, 252-266 (2006).
   40. WO2007/093412
   41. WO2003/095631
   42. Tedesco F S et al., Transplantation of genetically corrected human iPSC-derived progenitors in mice with limb-girdle muscular dystrophy. Sci Transl Med. 2012 Jun. 27; 4(140):140ra89.
   43. C. M. Kelly et al., Neonatal desensitization allows long-term survival of neural xenotransplants without immunosuppression. Nat Methods 6, 271-273 (2009).

1. Method for obtaining mesoangioblast (MAB)-like mesodermal stem/progenitor cells from pluripotent stem cells comprising the following steps:
(a) dissociating colonies of said cells to a first single cell suspension;
(b) seeding the first single cell suspension on a solid support coated with a cell culture substrate with appropriate culture medium and temperature in a low O₂ atmosphere to get a first cell culture;
(c) dissociating the cell culture to get a second single cell suspension;
(d) seeding the second single cell suspension as in step b) to get a second cell culture;
wherein steps (c) and (d) are optionally repeated.

2. The method according to claim 1 wherein said pluripotent stem cells are induced pluripotent cells (iPSCs).

3. The method according to claim 1 wherein said pluripotent stem cells are embryonic stem cells.

4. The method according to any of the previous claims wherein the low O₂ atmosphere of step b) is 3-5% O₂.

5. The method according to any of the previous claims wherein the dissociating steps a) and c) are performed in a mild non enzymatic environment.

6. The method according to claim 5 wherein the mild non enzymatic environment consists in incubating the culture with an EDTA-dissociation medium and a gentle shaking or scraping thereof.

7. The method according to any of the previous claims further comprising the following steps:
(e) dissociating the second cell culture by trypsinization to get a third cell suspension; and
(f) seeding said third cell suspension at an approximately 80% confluency as in step (b) up to an approximately full confluency in an appropriate MAB medium.

8. The method according to claim 8 further comprising the steps of:
(g) splitting the approximately full confluent cells to an approximately 80% confluency,
(h) culturing the cells as in (f),
optionally comprising a step of purification between step g) and h).

9. The method according to any of the previous claims wherein the mesoangioblast (MAB)-like mesodermal stem/progenitor cells are of human or any further mammalian origin.

10. The method according to any of claim 1, 2 or 4-9 wherein the iPSCs are isolated from healthy subject or from a muscular dystrophy subject.

11. The method according to claim 10 wherein the iPSCs are obtained by: (i) reprogramming skeletal muscle cells isolated from limb-girdle muscular dystrophy 2D (LGMD2D) subjects or (ii) genetically correcting Duchenne muscular dystrophy (DMD)-iPSCs with a human artificial chromosome containing the entire dystrophin locus (DYS-HAC).

12. The method according to any previous claim wherein the substrate with which the solid support is coated is a gelatinous mixture of extracellular proteins.

13. The method according to any previous claim wherein the solid support is Matrigel™-coated.

14. The method according to any previous claim wherein the cells are further negatively sorted once, twice or more than twice for SSEA1 and/or positively sorted for one, two or all three of CD34, Sca1 and CD44.

15. Mesoangioblast (MAB)-like mesodermal stem/progenitor cells obtainable or obtained with the method of any of previous claims.

16. Mesoangioblast (MAB)-like mesodermal stem/progenitor cells according to claim 15 terminally differentiable to skeletal myoblast or myocyte cells.

17. Mesoangioblast (MAB)-like mesodermal stem/progenitor cells according to claim 16, which are obtained by transduction with a vector able to express MyoD.

18. Mesoangioblast (MAB)-like mesodermal stem/progenitor cells according to claim 16 or 17 being transduced with a vector able to express the human alpha-sarcoglycan.

19. Mesoangioblast (MAB)-like mesodermal stem/progenitor cells according to any one of claims 15-18 for use as a medicament.

20. Mesoangioblast (MAB)-like mesodermal stem/progenitor cells according to any one of claims 15-18 for use in the treatment of a muscular dystrophy.

21. Mesoangioblast (MAB)-like mesodermal stem/progenitor cells according to claim 20 wherein the muscular dystrophy is LGMD2D or DMD.

22. Mesoangioblast (MAB)-like mesodermal stem/progenitor cells according to any one of claims 15-18 for use in gene therapies.

23. Method of treatment of a muscular dystrophy comprising the administration of a therapeutically effective amount of the cells according to any one of claims 15-18 to a subject in need thereof.

24. Method of treatment according to claim 23 wherein the cells are autologous.

25. Method of treatment according to claim 23 or 24 wherein the muscular dystrophy is LGMD2D or DMD.

26. Method for obtaining mesoangioblast (MAB)-like mesodermal stem/progenitor cells from pluripotent stem cells.

27. Use of pluripotent stem cells to obtain (MAB)-like mesodermal stem/progenitor cells.

28. The method of claim 26 or the use of claim 27 wherein said pluripotent stem cells are induced pluripotent stem cells (iPSCs) or embryonic stem cells.

29. The method or use of any one of claims 26 to 28 wherein said cells are human cells.


SEQUENCE LISTING (32 sequences)

SEQ ID NO: 1 - DNA, 22 bp, Artificial Sequence
  PRIMER SEQUENCES
cagggctgggagctgggttctg

SEQ ID NO: 2 - DNA, 18 bp, Artificial Sequence
  PRIMER SEQUENCES
cccagggccttgatgcct

SEQ ID NO: 3 - DNA, 24 bp, Artificial Sequence
  PRIMER SEQUENCES
gctatcaggacatagcgttggcta

SEQ ID NO: 4 - DNA, 40 bp, Artificial Sequence
  PRIMER SEQUENCES
gagaaaaggaggatcatggattcaagaaataaatgtaacg

SEQ ID NO: 5 - DNA, 28 bp, Artificial Sequence
  PRIMER SEQUENCES
tggcccctgctaactttctctcttagca

SEQ ID NO: 6 - DNA, 27 bp, Artificial Sequence
  PRIMER SEQUENCES
tggtatccacaacataaaatacgctaa

SEQ ID NO: 7 - DNA, 38 bp, Artificial Sequence
  PRIMER SEQUENCES
cctaagagtcactttctccatttacacagtgaagtgcc

SEQ ID NO: 8 - DNA, 20 bp, Artificial Sequence
  PRIMER SEQUENCES
gcctccacttctgtcttgct

SEQ ID NO: 9 - DNA, 20 bp, Artificial Sequence
  PRIMER SEQUENCES
ccaccaagaagtcacggtct

SEQ ID NO: 10 - DNA, 20 bp, Artificial Sequence
  PRIMER SEQUENCES
cactcaagcgctgcacgtcg

SEQ ID NO: 11 - DNA, 21 bp, Artificial Sequence
  PRIMER SEQUENCES
ggccgctgtagtccatcatgc

SEQ ID NO: 12 - DNA, 20 bp, Artificial Sequence
  PRIMER SEQUENCES
ccaggggtgcccagcgaatg

SEQ ID NO: 13 - DNA, 21 bp, Artificial Sequence
  PRIMER SEQUENCES
agccgtgagcagatgatcccc

SEQ ID NO: 14 - DNA, 20 bp, Artificial Sequence
  PRIMER SEQUENCES
ttcaccaccatggagaaggc

SEQ ID NO: 15 - DNA, 20 bp, Artificial Sequence
  PRIMER SEQUENCES
ggcatggactgtggtcatga

SEQ ID NO: 16 - DNA, 22 bp, Artificial Sequence
  PRIMER SEQUENCES
atgcacaacgagaggattttga

SEQ ID NO: 17 - DNA, 22 bp, Artificial Sequence
  PRIMER SEQUENCES
ctttgtgttcccaattccttcc

SEQ ID NO: 18 - DNA, 22 bp, Artificial Sequence
  PRIMER SEQUENCES
ttacctcttcctcccactccag

SEQ ID NO: 19 - DNA, 22 bp, Artificial Sequence
  PRIMER SEQUENCES
gggttttctccatgctgtttct

SEQ ID NO: 20 - DNA, 22 bp, Artificial Sequence
  PRIMER SEQUENCES
acccacacaggtgagaaacctt

SEQ ID NO: 21 - DNA, 22 bp, Artificial Sequence
  PRIMER SEQUENCES
gttgggaacttgaccatgattg

SEQ ID NO: 22 - DNA, 21 bp, Artificial Sequence
  PRIMER SEQUENCES
accattggcaatgagcggttc

SEQ ID NO: 23 - DNA, 22 bp, Artificial Sequence
  PRIMER SEQUENCES
cacttcatgatggagttgaagg

SEQ ID NO: 24 - DNA, 24 bp, Artificial Sequence
  PRIMER SEQUENCES
ccctttttctggagactaaataaa

SEQ ID NO: 25 - DNA, 18 bp, Artificial Sequence
  PRIMER SEQUENCES
cggtcacgaactccagca

SEQ ID NO: 26 - DNA, 20 bp, Artificial Sequence
  PRIMER SEQUENCES
acaagcagaagaacggcatc

SEQ ID NO: 27 - DNA, 19 bp, Artificial Sequence
  PRIMER SEQUENCES
ccatcttccaggagcgaga

SEQ ID NO: 28 - DNA, 20 bp, Artificial Sequence
  PRIMER SEQUENCES
tgtcataccaggaaatgagc

SEQ ID NO: 29 - DNA, 18 bp, Artificial Sequence
  PRIMER SEQUENCES
ggcacacgtggcttttcg

SEQ ID NO: 30 - DNA, 24 bp, Artificial Sequence
  PRIMER SEQUENCES
ggtgaacctcgtaagtttatgcaa

SEQ ID NO: 31 - DNA, 21 bp, Artificial Sequence
  PRIMER SEQUENCES
cagggcagtattctcgatatc

SEQ ID NO: 32 - DNA, 20 bp, Artificial Sequence
  PRIMER SEQUENCES
gcattggggtatcttggtac